(19) United States
(12) Patent Application Publication
(10) Pub. No.: US 2020/0147060 A1
(43) Pub. Date: May 14, 2020

(54) COMPOSITIONS AND METHODS OF TREATING CANCER

(71) Applicant: UNIVERSITY OF IOWA RESEARCH FOUNDATION, Iowa City, IA (US)

(72) Inventors: Michael K. Schultz, Iowa City, IA (US); Somya Kapoor, Iowa City, IA (US); Douglas R. Spitz, Iowa City, IA (US); Andrean Burnett, Iowa City, IA (US); Ling Yang, Iowa City, IA (US)

(73) Assignee: UNIVERSITY OF IOWA RESEARCH FOUNDATION, Iowa City, IA (US)

(21) Appl. No.: 16/668,736

(22) Filed: Oct. 30, 2019

Related U.S. Application Data
(60) Provisional application No. 62/752,603, filed on Oct. 30, 2018.

(51) Int. Cl. A61K 31/437; A61K 31/197; A61K 31/192; A61K 31/472; A61P 35/00

(52) U.S. Cl. CPC A61K 31/437; A61K 31/197; A61P 35/00; A61K 31/472; A61K 31/192

(57) ABSTRACT

The invention provides in certain embodiments a therapeutic regimen comprising (a) an anti-cancer combination of vemurafenib and cobimetinib, or a combination of trametinib and dabrafenib, and (b) a secondary agent comprising L-buthionine-[S,R]-sulfoximine (BSO), or phenyl butyric acid (PBA) or a pharmaceutically acceptable salt thereof, and chloroquine or hydrochloroquine (HCQ) for the therapeutic treatment of a hyperproliferative disorder.

### RELATED APPLICATION

This application claims priority to U.S. Provisional Patent Application No. 62/752,603 that was filed on Oct. 30, 2018. The entire content of the application referenced above is hereby incorporated by reference herein.

### FEDERAL GRANT SUPPORT

This invention was made with government support under CA172218 awarded by the National Institutes of Health. The government has certain rights in the invention.

### BACKGROUND

Melanoma is a cancer of the skin and is the fastest growing cancer incidence in the world today. Disease detected early can be removed by surgery, but when melanoma spreads to other parts of the body (called metastatic melanoma) it is almost uniformly fatal. The reason for this is that metastatic melanoma is either inherently resistant or rapidly becomes resistance to all forms of treatment. The first new therapy that appeared effective for melanoma was approved in 2011. The pharmaceutical called vemurafenib targets patients with a gene mutation (BRAF^{V600E}) that is present in about half of melanoma patients. Although these patients may respond well to the treatment, melanoma develops resistance to this therapy rapidly (often within months). Thus, the new therapy, which initially was heralded as the end of melanoma, extends life expectancy by only months with severe side effects. Vemurafenib is one of several BRAF inhibitors that are being used for melanoma therapy that target the BRAF protein. Melanoma develops resistance to all of these therapies. Several other drugs that have different mechanisms of action are also approved for melanoma treatment, but the disease eventually develops resistance to all therapies for melanoma. There is no treatment for metastatic melanoma that overcomes resistance of melanoma cancer cells, which leads to a high mortality rate.

Thus, there is a continuing need for compositions and methods for the treatment of melanoma in animals (e.g., humans). Combination therapies that overcome resistance mechanisms that arise in almost all melanoma patients are particularly needed.

### SUMMARY

In certain embodiments, the present invention provides a therapeutic regimen comprising (a) an anti-cancer BRAF inhibitor agent or combination of BRAF inhibitor and MEK inhibitor, and (b) a secondary agent comprising L-buthionine-[S,R]-sulfoximine (B SO), or combination of phenylbutyric acid (PBA) or a pharmaceutically acceptable salt thereof, and chloroquine or hydrochloroquine (HCQ) for the therapeutic treatment of a hyperproliferative disorder.

In certain embodiments, the present invention provides a therapeutic regimen consisting essentially of (a) an anti-cancer combination of vemurafenib and cobimetinib, or a combination of trametinib and dabrafenib, and (b) a secondary agent comprising L-buthionine-[S,R]-sulfoximine (BSO), or phenyl butyric acid (PBA) or a pharmaceutically acceptable salt thereof, and chloroquine or hydrochloroquine (HCQ) for the therapeutic treatment of a hyperproliferative disorder.

As a combined treatment the combination treatment effectively destroys metastatic melanoma cancer cells. In certain embodiments, the hyperproliferative disorder is cancer. In certain embodiments, the cancer is drug-resistant. As used herein, the term “drug-resistant” is reduction in effectiveness of a drug in killing malignant cells; reducing cancerous tumor size and rate of growth; and ameliorating the disease or condition. In certain embodiments, the drug's effectiveness is reduced by at least about 10%, 20%, 30%, 40%, 50%, 60%, 70%, 80%, 90%, or even 100%, as compared to its effects when first administered to the mammal.

In certain embodiments, the present invention provides the use of a therapeutic combination comprising (a) an anti-cancer combination of vemurafenib and cobimetinib, or a combination of trametinib and dabrafenib, and (b) a secondary agent comprising L-buthionine-[S,R]-sulfoximine (BSO), or phenyl butyric acid (PBA) or a pharmaceutically acceptable salt thereof, and chloroquine or hydrochloroquine (HCQ), for the therapeutic treatment of a hyperproliferative disorder in a mammal.

In certain embodiments, the present invention provides the use of a therapeutic combination consisting essentially of (a) an anti-cancer combination of vemurafenib and cobimetinib, or a combination of trametinib and dabrafenib, and (b) a secondary agent comprising L-buthionine-[S,R]-sulfoximine (BSO), or phenyl butyric acid (PBA) or a pharmaceutically acceptable salt thereof, and chloroquine or hydrochloroquine (HCQ), for the therapeutic treatment of a hyperproliferative disorder in a mammal.

In certain embodiments, the present invention provides a kit comprising a therapeutic combination comprising (a) an anti-cancer combination of vemurafenib and cobimetinib, or a combination of trametinib and dabrafenib, and (b) a secondary agent comprising L-buthionine-[S,R]-sulfoximine (BSO), or phenylbutyric acid (PBA) or a pharmaceutically acceptable salt thereof, and chloroquine or hydrochloroquine (HCQ), a container, and a package insert or label indicating the administration of the therapeutic combination for treating a hyperproliferative disorder.

In certain embodiments, the present invention provides a kit comprising a therapeutic combination consisting essentially of (a) an anti-cancer combination of vemurafenib and cobimetinib, or a combination of trametinib and dabrafenib, and (b) a secondary agent comprising L-buthionine-[S,R]-sulfoximine (B SO), or phenyl butyric acid (PBA) or a pharmaceutically acceptable salt thereof, and chloroquine or hydrochloroquine (HCQ), a container, and a package insert or label indicating the administration of the therapeutic combination for treating a hyperproliferative disorder.

In certain embodiments, the present invention provides a product comprising a therapeutic combination comprising (a) an anti-cancer combination of vemurafenib and cobimetinib, or a combination of trametinib and dabrafenib, and (b) a secondary agent comprising L-buthionine-[S,R]-sulfoximine (BSO), or phenyl butyric acid (PBA) or a pharmaceutically acceptable salt thereof, and chloroquine or hydrochloroquine (HCQ) as a combined preparation for separate, simultaneous or sequential use in the treatment of a hyperproliferative disorder.

In certain embodiments, the present invention provides a product comprising a therapeutic combination consisting essentially of (a) an anti-cancer combination of vemurafenib and cobimetinib, or a combination of trametinib and dabrafenib, and (b) a secondary agent comprising L-buthionine-[S,R]-sulfoximine (BSO), or phenyl butyric acid (PBA) or a pharmaceutically acceptable salt thereof, and chloroquine or hydrochloroquine (HCQ) as a combined preparation for separate, simultaneous or sequential use in the treatment of a hyperproliferative disorder.

In certain embodiments, the present invention provides a method for treating a hyperproliferative disorder in a mammal, comprising administering to the mammal a therapeutic combination comprising (a) an anti-cancer combination of vemurafenib and cobimetinib, or a combination of trametinib and dabrafenib, and (b) a secondary agent comprising L-buthionine-[S,R]-sulfoximine (BSO), or phenyl butyric acid (PBA) or a pharmaceutically acceptable salt thereof, and chloroquine or hydrochloroquine (HCQ) as a combined preparation for separate, simultaneous or sequential use in the treatment of a hyperproliferative disorder.

In certain embodiments, the present invention provides a method for treating a hyperproliferative disorder in a mammal, comprising administering to the mammal a therapeutic combination consisting essentially of (a) an anti-cancer combination of vemurafenib and cobimetinib, or a combination of trametinib and dabrafenib, and (b) a secondary agent comprising L-buthionine-[S,R]-sulfoximine (B SO), or phenyl butyric acid (PBA) or a pharmaceutically acceptable salt thereof, and chloroquine or hydrochloroquine (HCQ) as a combined preparation for separate, simultaneous or sequential use in the treatment of a hyperproliferative disorder.

In certain embodiments, the present invention provides a use of (a) an anti-cancer combination of vemurafenib and cobimetinib, or a combination of trametinib and dabrafenib, and (b) a secondary agent comprising L-buthionine-[S,R]-sulfoximine (BSO), or phenyl butyric acid (PBA) or a pharmaceutically acceptable salt thereof, and chloroquine or hydrochloroquine (HCQ) for the therapeutic treatment of a hyperproliferative disorder.

In certain embodiments, the present invention provides a method of depleting glutathione (GHS) in a cancer cell in a patient comprising administering to the patient buthionine sulfoximine (BSO), wherein the cancer cell is determined to be resistant to BRAF inhibitors (BRAFᵢ).

In certain embodiments, the present invention provides a method of inhibiting endoplasmic reticulum (ER)-stress and autophagy in a cancer cell in a patient comprising administering to the patient buthionine sulfoximine (BSO), wherein the cancer cell is determined to be resistant to BRAF inhibitors (BRAFᵢ).

In certain embodiments, the present invention provides a method of depleting glutathione (GHS) in a cancer cell in a patient comprising administering to the patient buthionine sulfoximine (BSO), wherein the cancer cell is determined to be resistant to mitogen-activated protein kinase enzyme inhibitors (MEKᵢ).

In certain embodiments, the present invention provides a method of inhibiting endoplasmic reticulum (ER)-stress and autophagy in a cancer cell in a patient comprising administering to the patient buthionine sulfoximine (BSO), wherein the cancer cell is determined to be resistant to mitogen-activated protein kinase enzyme inhibitors (MEKᵢ).

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1J. MAPKᵢ-induced changes in the cellular redox state plays a role in acquisition of resistance. Induction of resistance MAPKᵢₕ in A375 (BRAFV600E) human melanoma cells is accompanied by profound changes in O2 metabolism, increased parameters indicative of oxidative stress, depletion of total GSH, and is completely abrogated by inhibition of GSH synthesis. (A) The ratio of basal metabolic oxygen consumption (BMOC)/extracellular acidification rate (ECAR) in response to continuous exposure to 5 μM Vem (N=6) as determined by Seahorse metabolic profiling; (B) Measurement of cellular pro-oxidant levels as determined by dyhdroethidium (DHE) oxidation in response to 5 μM Vem (N=3); (C) GSSG:GSH in response to Vem (N=2); (D) total cellular GSH in response to Vem (N=2). (E) Clonogenic survival of A375 BRAFV600E melanoma cells in response to Vem (5 μM) with and without BSO (0.5 mM) (N=2, n=6); (F) clonogenic survival of A375 BRAFV600E melanoma cells in response to Vem (5 μM) with and without BSO (0.5 mM) (N=2, n=6). (G-J) Inhibition of GSH synthesis using BSO in combination with MAPKᵢ promotes apoptosis in A375 melanoma cell lines. (G) Annexin V flow cytometry analysis of A375 BRAFV600E melanoma cells over 10 days treatment with Vem; and (H) Vem+Cobi (with and without the inclusion of BSO; N=6); (I, J) Gated flow cytometry signal intensity analysis showing a significant increase in Annexin V positive (apoptotic) cells with the inclusion of BSO relative to MAPKᵢ alone as cells adapt to MAPKᵢ treatments (N=6). ±SEM; **p=0.001 to 0.01, ****p<0.0001.

FIGS. 2A-2D. Prolonged MAPKᵢ induces protective ER-stress responses in melanoma cells that can be attenuated by inhibiting synthesis of glutathione. Inhibition of GSH synthesis attenuates ER-stress responses induced by MAPKᵢₕ (Vem 5 μM; Cobi 0.1 μM). (A-C) Prolonged MAPKᵢ induces increase in ER-stress markers that subsequently return to lower levels as cells become resistant. (A) BiP expression by flow cytometry in response to Vem (N=6); (B) p-eIF2α:eIF2α (western blot) in response to Vem (N=2); (C) BiP expression in response to Vem+Cobi (N=3); (D) BiP expression (by flow) is significantly attenuated by inclusion of BSO (0.5 mM) with Vem (N=3). ±SEM; *p=0.01 to 0.05, ****P<0.0001.

FIGS. 3A-2F. Prolonged MAPKᵢ induces increases in autophagic flux in melanoma cells that can be diminished by inhibiting synthesis of glutathione. Inhibition of GSH synthesis attenuates autophagic flux induced by MAPKᵢₕ (Vem 5 μM; Cobi 0.1 μM) (A-D) Prolonged MAPKᵢ induces an increase in autophagy that subsequently return to lower levels as cells become resistant. (A) Area fraction covered by autophagosomes quantified by Transmission Electron Microscopy (TEM) in response to Vem (n=64); (B) LC3B-II expression (western blot) in response to Vem (N=2); (C) autophagic flux measured by flow cytometry in response to Vem (N=6); (D) autophagic flux in response to Vem+Cobi (N=6). (E, F) autophagic flux is significantly attenuated by inclusion of BSO (0.5 mM) with MAPKᵢₕ; (E) Vem (N=6); and (F) Vem+Cobi (N=6). ±SEM; *p=0.01 to 0.05, **p=0.001 to 0.01 ****P<0.0001.

FIG. 4A-4H. Simultaneous Attenuation of UPR and Inhibition of Autophagic Flux Prevents Resistance to MAPKᵢₕ In vitro. (A-H) Simultaneous attenuation of UPR and inhibition of autophagic flux prevents resistance to MAPKᵢₕ in Vitro. (A) Autophagic flux quantified in response to combination of PBA (2 mM) and HCQ (12 μM) with Vem (5 μM) in A375 melanoma cells (N=6); (B) autophagic flux quantified in response to combination of PBA (2 mM) and HCQ (12 μM) with Vem+Cobi in A375 melanoma cells (N=6); (C,D) clonogenic survival of A375 cells when treated with Vem alone or in combination with PBA and HCQ (N=2,n=6); (E,F) percent of apoptotic when treated with the combination of PBA and HCQ in combination with MAPKᵢₕ; (E) Vem (N=6); and (F) Vem+Cobi (N=6). (G) Gated flow cytometry signal intensity analysis showing a significant increase in Annexin V positive (apoptotic) cells with the inclusion of PBA and HCQ relative to Vem alone as cells adapt to Vem treatment (N=6). (H) Gated flow cytometry signal intensity analysis showing a significant increase in Annexin V positive (apoptotic) cells with the inclusion of PBA and HCQ relative to Vem+Cobi as cells adapt to MAPKᵢ (N=6). ±SEM; *p=0.01 to 0.05, **p=0.001 to 0.01 ****P<0.0001.

FIGS. 5A-5F. Pharmacologically Inhibiting Autophagy and ER-stress Simultaneously in Combination with MAPKᵢₕ Improve Overall Survival in Vivo. Simultaneous inhibition of autophagy and ER-stress with MAPKᵢₕ (Vem and Vem+Cobi) improves outcome in melanoma tumor bearing mice. (A-D) Simultaneous inhibition of ER-stress using PBA (250 mg kg⁻¹ twice a day) and autophagic flux using (HCQ 10 mg kg⁻¹ twice a day) with MAPKᵢₕ (Vem 16 mg kg⁻¹ twice a day; Cobi 1 mg kg⁻¹ once a day) improves outcome in mice bearing MAPKᵢ-sensitive A375 melanoma xenografts (N=13). (A) estimated tumor growth curves in response to PBA and HCQ with Vem; (B) estimated tumor growth curves in response to PBA and HCQ with Vem+Cobi; (C) overall survival in response to PBA and HCQ with Vem; (D) overall survival in response to PBA and HCQ with Vem+Cobi. (E, F) Simultaneous inhibition of ER-stress using PBA (250 mg kg⁻¹ twice a day) and autophagic flux using (HCQ 6.6 mg kg⁻¹ twice a day) with MAPKᵢₕ (Vem 10 mg kg⁻¹ twice a day) improves outcome in mice bearing MAPKᵢ-resistant 451LuBR melanoma xenografts (N=10); (E) estimated tumor growth curves in response to PBA and HCQ with Vem; and (F) overall survival in response to PBA and HCQ with Vem.

FIGS. 6A-6H. Acquisition of resistance to MAPKi is accompanied by changes in oxidative metabolism of melanoma cells. (A) Normalized clonogenic survival of A375 (N=2, n=3) and 451Lu (N=1,n=3) cells when treated continuously with Vemurafenib (5 μM) alone and in combination with Cobimetinib (0.1 μM). Changes in the BMOC/ECAR ratio of A375 (N=2, n-6) and 451Lu (N=2, n=6) cells as determined by the Seahorse XF6 cell mitochondrial stress test when treated continuously with; (B) Vemurafenib (5 μM); and (C) Vemurafenib (5 μM) in combination with Cobimetinib (0.1 μM). (D) Changes in the normalized mean fluorescence intensity of Mitosox when A375 (N=6) and 451 Lu (N=3) cells were treated with Vemurafenib+Cobimetinib. (E) Changes in the normalized mean fluorescence intensity (MFI) of Mitosox when A375 (N=3) cells were treated with Vemurafenib. (F) Changes in the normalized MFI of DHE due to oxidation in response to continuous treatment of A375 cells with vemurafenib (N=3). (G) Concentration of reduced GSH in A375 cells during continuous treatment with Vemurafenib (N=3). (H) Percent of GSH as GSSG in A375 cells when treated with Vemurafenib (N=3). ±SEM; *p=0.01 to 0.05, **p=0.001 to 0.01 ****P<0.0001.

FIGS. 7A-7L. Reduced Thiols Play a Vital Role in Development of Resistance to MAPKi. (A-E) Reduced thiols play a vital role in development of resistance to MAPKi in vitro. Changes in (A) cellular concentration of reduced glutathione (GSH, nmoles/mg protein) (B) cellular concentration of oxidized glutathione (GSSG, nmoles/mg protein) of A375 melanoma cells when treated with buthionine sulfoximine (BSO, 0.5 mM) and 2-mercaptoethanol (ME, 150 μM) in presence of vemurafenib (Vem, 5 μM) (N=3). Difference in clonogenic survival of A375 (N=2, n=3) and 451 Lu (N=1, n=3) melanoma cells when treated with vem alone compared to (C) vem in combination with ME and (D) vem in combination with BSO. (E) Addition of ME to the combination of vem and BSO rescues clonogenic survival in A375 and 451 Lu melanoma cells. (F-L) Depletion of reduced GSH in vivo prevents development of resistance to vemurafenib in A375 xenograft bearing Athymic nude mice. Spider plots show individual tumor growth rates in A375 tumor bearing mice (N=8) when (F) untreated; (G) treated with BSO (15 mM in drinking water); (H) treated with AIN-76A rodent diet mixed with vemurafenib (PLX4720); (I) combination of AIN-76A rodent diet mixed with vemurafenib (PLX4720) with BSO. (J) Levels of total cellular GSH in tumor tissue obtained from untreated mice and mice treated with BSO. (K) Progression Free Survival (PFS) and (L) Overall survival (OS). ±SEM; *p=0.01 to 0.05, **p=0.001 to 0.01 ****P<0.0001.

FIGS. 8A-8J. Simultaneous Inhibition of Autophagic Flux and Attenuation of UPR Prevents Development of Resistance to MAPKi in Vivo and in Vitro. (A-C) Simultaneous inhibition of autophagy and attenuation of UPR using Hydroxychloroquine (HCQ, 12 μM) and Sodium phenyl butyrate (PBA, 2 mM) respectively prevents development of resistance to MAPKi in vitro. Clonogenic survival of A375 cells in response to simultaneous treatment with HCQ and PBA. (A) in combination with MAPKi (N=2,n=3). (B) Percent of non-apoptotic (Annexin V negative), live (Hoechst negative) A375-CG-LC3B cells when treated with MAPKi alone and in combination with HCQ and PBA (N=6). (C) Autophagic flux (MFI mcherry: MFI GFP) quantified for the non-apoptotic live sub population of A375-CG-LC3B cells when treated with MAPKi alone and in combination with HCQ and PBA (N=6). (d-1) Pharmacologic simultaneous inhibition of autophagy and UPR combination with MAPKih improves outcome In-Vivo. Spider plots show individual tumor growth rates in athymic nude mice bearing MAPKi-sensitive A375 xenografts (N=13) when (D) untreated; (E) treated with MAPKi (Vem 16 mg kg⁻¹ twice a day; Cobi 1 mg kg⁻¹ once a day); and (F) treated with a combination of MAPKi with HCQ (10 mg kg⁻¹ twice a day) and PBA (250 mg kg⁻¹ twice a day). (G) Overall survival (OS) of mice in these treatment groups. (H) Athymic nude mice bearing MAPKᵢ-resistant 451LuBR melanoma xenografts; (I) estimated tumor growth rates in mice treated with MAPKi alone (Vem 10 mgkg⁻¹ twice a day) and in combination with PBA (250 mg kg⁻¹ twice a day) and (HCQ 6.6 mg kg⁻¹ twice a day); and (J) overall survival of mice in these groups (N=10). ±SEM; *p=0.01 to 0.05, **p=0.001 to 0.01 ****P<0.0001.

### DETAILED DESCRIPTION

Melanoma is the dangerous type of skin cancer that develops in cells that produce melanin (melanocytes), usually presenting as an irregular spot/mole on the skin. Causes of melanoma include UV radiation and a genetic predisposition to this type of cancer. Unlike other cancers, prevalence of melanoma is increasing, with the highest occurrence among individuals 25-29 years old. The overall lifetime risk of developing melanoma is 2.4%. In 2015, 73,870 new invasive melanomas are expected to be diagnosed, with 9,940 people expected to die of melanoma. With early treatment, survival rate is 97%.

Melanoma can migrate to other parts of the body (metastatic melanoma), and one year survival rate drastically decreases with metastasis—15-20% for Stage IV. Current types of treatment include surgery, immunotherapy (Immune checkpoint inhibitors for advanced melanoma), chemotherapy, radiation therapy, targeted therapy (target cells with gene changes) and BRAF Inhibitors. BRAF is a protein kinase of the mitogen-activated protein kinase (MAPK) pathway, and it regulates cell growth, proliferation, and differentiation. Research suggests a BRAF^{V600E} mutation causes the BRAF protein (produced through the MAPK pathway) to become oncogenic. The mutation may lead to increased and uncontrolled cell proliferation, and resistance to apoptosis. The BRAF mutation is observed in about 50% of melanoma tumors. Its presence is associated with poor prognosis in metastatic melanoma.

Melanoma is the fastest growing cancer incidence in the United States. Surgery is curative for melanoma confined to the skin, but metastatic melanoma is lethal. Current FDA approved therapies for metastatic melanoma (e.g., Vemurafenib, Ipilimumab), have increased life expectancy by months, however resistance develops rapidly. The exact mechanism by which drug resistance develops is unclear; however, autophagy is known to play a major role. Autophagy is a self-degradative response of the cell towards nutrient stress. Conversely, autophagy also plays a housekeeping role by removing mis-folded or aggregated proteins and clearing damaged organelles by forming autophagosomes. Thus, autophagy is believed to play an important role in tumor progression and developing drug resistance during later stages of cancer. The Unfolded Protein Response (UPR) mediated by GRP78 ER associated protein degradation is one of the pathways that initiates autophagy in stressed cells. UPR involves the activation of three signaling pathways mediated by IRE-1, PERK and ATF6. These pathways work towards decreasing the protein load of ER by increasing the expression of molecular chaperons, activation of ERAD (ER associated protein degradation) and autophagy. However if the damage caused by the stress is extensive UPR signaling pathways initiate apoptosis. Emerging evidence shows that in malignant cells ER-stress can be pro-survival and contribute to the development of drug resistance by initiating autophagy.

Melanoma resistance to MAPK inhibitors (MAPKᵢₕ) has been attributed to drug induced activation of ER-stress responses that stimulate increased autophagic flux. In this example, it is shown that adaptation to MAPKᵢ is accompanied by 70% depletion of glutathione (GSH) and a 30-fold increase in oxidized glutathione (GSSG) indicating that MAPKᵢ are inducing profound thiol-mediated metabolic oxidative stress. It is further shown that pharmacologic depletion of glutathione (GSH) in melanoma cells, using GSH-synthesis inhibitor buthionine sulfoximine (BSO), completely inhibits acquisition of resistance to MAPKᵢ by preventing ER-stress responses and inhibiting autophagic flux. Finally, combining MAPKᵢ with simultaneous inhibition of autophagic flux and attenuation of ER-stress responses inhibited acquisition of MAPKᵢₕ resistance in melanoma tumors in mice and significantly enhanced overall survival (20% complete remissions). These results show that simultaneous targeting GSH metabolism, ER-stress, and autophagy provides a novel biochemical paradigm for abrogating the development of resistance to MAPKᵢₕ therapy in malignant melanoma.

Attempts to overcome melanoma resistance to MAPKᵢₕ by inhibiting autophagic flux or ER-stress pathways alone have been unsuccessful clinically. Here, GSH is shown to play a central role in activating these protective pathways, as well as providing resistance to BRAFᵢ-induced oxidative stress. A GSH synthesis inhibitor (BSO) prevented acquisition of resistance to BRAF inhibitors (BRAFᵢₕ) in melanoma cells. Furthermore, MAPKᵢₕ combined with simultaneous inhibition of autophagic flux and attenuation of ER-stress can prevent the acquisition of resistance melanoma tumors in mice to MAPKᵢₕ. Thus, the results presented here provide new combination therapies for metastatic melanoma that can prevent the acquisition of resistance to MAPKᵢ and improve outcomes for metastatic melanoma patients.

A new combination therapy regimen has been developed that kills vemurafenib-resistant cells, but is virtually non-toxic to the rest of the body.

### Anti-Cancer Agents

As used herein, the term “anti-cancer agent” includes therapeutic agents that kill cancer cells; slow tumor growth and cancer cell proliferation; and ameliorate or prevent one or more of the symptoms of cancer. For example, the term “anti-cancer agent” includes vemurafenib, cobimetinib, trametinib and dabrafenib.

Vemurafenib (Zelboraf®) is a cancer growth blocker and is a treatment for advanced melanoma.

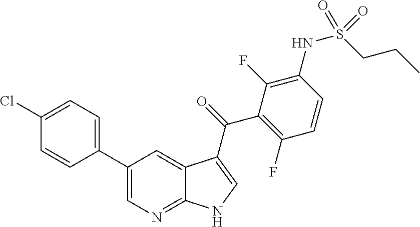 [SMILES: CCCS(=O)(=O)Nc1ccc(F)c(C(=O)c2c[nH]c3ncc(-c4ccc(Cl)cc4)cc23)c1F]

Vemurafenib stops the proliferative effects of oncogenic BRAF protein (i.e., is a BRAF inhibitor). The standard method of administration is an oral tablet, administered 4× daily. Unfortunately, metastatic melanoma can resist vemurafenib treatment. Vemurafenib slows tumor progression for only about 7.2 months. As a result, finding an effective treatment for metastatic melanoma is challenging.

Cobimetinib (Cotellic®) is a MEK inhibitor.

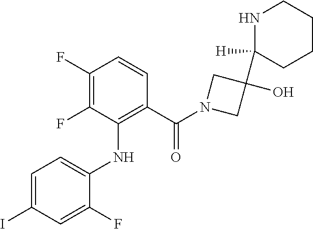 [SMILES: O=C(c1ccc(F)c(F)c1Nc1ccc(I)cc1F)N1CC(O)([C@@H]2CCCCN2)C1]

Cobimetinib is used in combination with vemurafenib to treat melanoma. The combination of vemurafenib and cobimetinib increased progression-free survival to an average of 12.3 months, compared to 7.2 months for vemurafenib alone.

Trametinib (Mekinist®) is an oral MEK inhibitor.

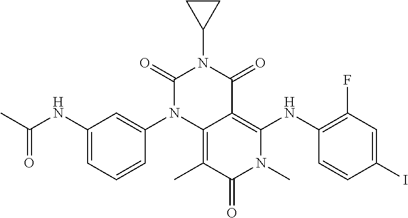 [SMILES: CC(=O)Nc1cccc(-n2c(=O)n(C3CC3)c(=O)c3c(Nc4ccc(I)cc4F)n(C)c(=O)c(C)c32)c1]

Trametinib is a targeted therapy that targets the MEK 1 and MEK 2 protein (kinase) within the cancer cell. It is usually given in combination with a BRAF kinase inhibitor.

Dabrafenib (Tafinlar®) is an oral BRAF kinase inhibitor.

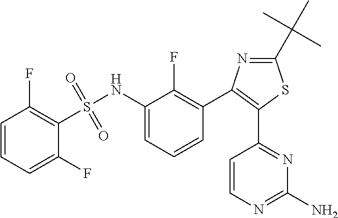 [SMILES: CC(C)(C)c1nc(-c2cccc(NS(=O)(=O)c3c(F)cccc3F)c2F)c(-c2ccnc(N)n2)s1]

Dabrafenib is a targeted therapy that targets the mutated BRAF proteins (kinase) within the cancer cell. Dabrafenib acts as an inhibitor of the associated enzyme B-Raf, which plays a role in the regulation of cell growth. Clinical trial data demonstrated that resistance to dabrafenib and other BRAF inhibitors occurs within 6 to 7 months. To overcome this resistance, the BRAF inhibitor dabrafenib is often combined with the MEK inhibitor trametinib.

Secondary Agents

### 1. Phenylbutyrate, and Salts Thereof

As used herein, the term “Phenylbutyrate and salts thereof” includes salts of Phenylbutyrate. PBA has the following structure:

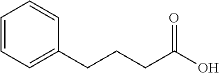 [SMILES: O=C(O)CCCc1ccccc1]

In certain embodiments, Phenylbutyrate is Buphenyl® (sodium phenylbutyrate). Sodium phenylbutyrate is used for chronic management of urea cycle disorders (UCDs). Its mechanism of action involves the quick metabolization of sodium phenylbutyrate to phenylacetate. Phenylacetate then conjugates with glutamine (via acetylation) to form phenylacetylglutamine, and phenylacetylglutamine is excreted by the kidneys. It has been observed that sodium phenylbutyrate reduces Endoplasmic Reticulum (ER) stress.

The cellular response to ER-stress is neither fully oncogenic nor completely tumor suppressive. It involves complex signaling with many pathways. The relative importance of each pathway varies between cells depending on chronicity of ER-stress, and on relative expression of various associated proteins. As solid cancers grow, nutrients and oxygen required exceed capacity of existing vascular bed, which can trigger angiogenesis (development of new blood vessels) to get more oxygen/nutrients to the cancers. Cancers, however, usually become hypoxic and nutrient-depleted, and with the hypoxia leading to impaired generation of ATP. The low ATP levels compromise ER protein folding which leads to ER-stress. Thus, unfolded, and/or misfolded proteins are associated with ER-stress and cancer cells exist with higher levels of ER-stress relative to health cells.

Potential outcomes as a consequence of ER-stress include high rates of protein synthesis that would trigger increased expression of autophagy, which is cytoprotective during stress (liberates amino acids, and removes damaged organelles). Another outcome would be an increased tolerance to hypoxia, which would promote tumor growth. This would also increase autophagy, promoting drug resistance. Thus, a successful treatment would inhibit autophagy and promote cell death.

Sodium phenylbutyrate decreases ER-stress. Lowering ER-stress prevents tolerance to hypoxia, and prevents cytoprotective autophagy (which leads to drug resistance). Phenylbutyrate acts as a “chemical chaperone,” meaning it guides proper protein folding, and the presence of properly folded proteins lowers ER-stress.

2. L-buthionine-[S,R]-sulfoximine (BSO)

L-buthionine-[S,R]-sulfoximine (BSO) is an inhibitor of glutathione. BSO inhibits gamma-glutamylcysteine synthetase, the enzyme required in the first step of glutathione synthesis.

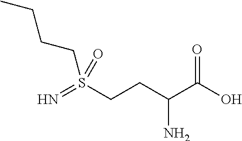

### 3. Chloroquine and Hydrochloroquine (HCQ)

Chloroquine and hydrochloroquine (HCQ) have long been used in the treatment or prevention of malaria. It is also known that hydroxychloroquine can act as an inhibitor of autophagic flux by inhibiting the fusion of lysozomes to autophagosomes.

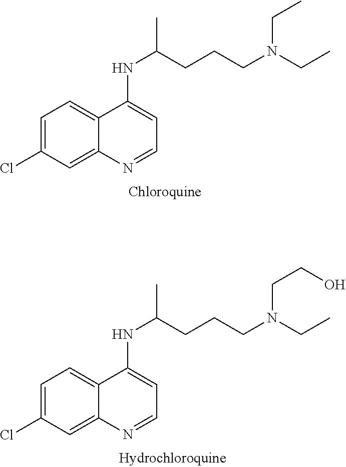 [SMILES: CCN(CC)CCCC(C)Nc1ccnc2cc(Cl)ccc12 ; CCN(CCO)CCCC(C)Nc1ccnc2cc(Cl)ccc12]

### Compositions and Methods of Administration

In certain embodiments, the present invention provides a therapeutic regimen comprising (a) an anti-cancer BRAF inhibitor agent or combination of BRAF inhibitor and MEK inhibitor, and (b) a secondary agent comprising L-buthionine-[S,R]-sulfoximine (BSO), or combination of phenylbutyric acid (PBA) or a pharmaceutically acceptable salt thereof, and chloroquine or hydrochloroquine (HCQ) for the therapeutic treatment of a hyperproliferative disorder.

In certain embodiments, the anti-cancer agent consists essentially of a BRAF inhibitor agent.

In certain embodiments, the anti-cancer agent consists essentially of a combination of a BRAF inhibitor agent and a MEK inhibitor.

In certain embodiments, the BRAF inhibitor agent is vemurafenib or dabrafenib.

In certain embodiments, the MEK inhibitor is cobimetinib or trametinib

In certain embodiments, the present invention provides a therapeutic regimen consisting essentially of (a) an anti-cancer combination of vemurafenib and cobimetinib, or a combination of trametinib and dabrafenib, and (b) a secondary agent comprising L-buthionine-[S,R]-sulfoximine (BSO), or phenyl butyric acid (PBA) or a pharmaceutically acceptable salt thereof, and chloroquine or hydrochloroquine (HCQ) for the therapeutic treatment of a hyperproliferative disorder.

In certain embodiments, the hyperproliferative disorder is cancer.

In certain embodiments, the cancer is melanoma.

In certain embodiments, the cancer is unresectable metastatic melanoma.

In certain embodiments, the melanoma is resistant to treatment with the anti-cancer combination of BRAF inhibitor and MEK inhibitor.

In certain embodiments, the melanoma is resistant to treatment with the anti-cancer combination of vemurafenib and cobimetinib.

In certain embodiments, the melanoma is resistant to treatment with the anti-cancer combination of trametinib and dabrafenib.

In certain embodiments, the secondary agent is administered simultaneously with the anti-cancer combination.

In certain embodiments, the secondary agent and the anti-cancer combination are administered sequentially.

In certain embodiments, the administration of the anti-cancer combination begins about 1 to about 10 days before administration of the secondary agent.

In certain embodiments, the administration of the secondary agent thereof begins about 1 to about 10 days before administration of the anti-cancer combination.

In certain embodiments, the administration of the secondary agent and administration of the anti-cancer combination begin on the same day.

In certain embodiments, the anti-cancer combination comprises vemurafenib and cobimetinib.

In certain embodiments, the anti-cancer combination comprises trametinib and dabrafenib.

In certain embodiments, the secondary agent comprises BSO.

In certain embodiments, the secondary agent comprises PBA or a pharmaceutically acceptable salt thereof and chloroquine or HCQ.

In certain embodiments, the secondary agent comprises PBA and HCQ.

In certain embodiments, the vemurafenib, cobimetinib, PBA and HCQ are administered in combination, and the cancer is melanoma.

In certain embodiments, the vemurafenib, cobimetinib, and BSO are administered in combination, and the cancer is melanoma.

In certain embodiments, the trametinib, dabrafenib, PBA and HCQ are administered in combination. In certain embodiments, the trametinib, dabrafenib, and BSO are administered in combination, and the cancer is melanoma.

In certain embodiments, the present invention provides the use of a therapeutic combination comprising (a) an anti-cancer BRAF inhibitor agent or combination of BRAF inhibitor and MEK inhibitor, and (b) a secondary agent comprising L-buthionine-[S,R]-sulfoximine (BSO), or combination of phenylbutyric acid (PBA) or a pharmaceutically acceptable salt thereof, and chloroquine or hydrochloroquine (HCQ), for the therapeutic treatment of a hyperproliferative disorder in a mammal.

In certain embodiments, the anti-cancer agent consists essentially of a BRAF inhibitor agent.

In certain embodiments, the anti-cancer agent consists essentially of a combination of a BRAF inhibitor agent and a MEK inhibitor.

In certain embodiments, the BRAF inhibitor agent is vemurafenib or dabrafenib.

In certain embodiments, the MEK inhibitor is cobimetinib or trametinib

In certain embodiments, the present invention provides the use of a therapeutic combination consisting essentially of (a) an anti-cancer combination of vemurafenib and cobimetinib, or a combination of trametinib and dabrafenib, and (b) a secondary agent comprising L-buthionine-[S,R]-sulfoximine (BSO), or phenyl butyric acid (PBA) or a pharmaceutically acceptable salt thereof, and chloroquine or hydrochloroquine (HCQ), for the therapeutic treatment of a hyperproliferative disorder in a mammal.

In certain embodiments, the present invention provides a kit comprising a therapeutic combination comprising (a) an anti-cancer combination of vemurafenib and cobimetinib, or a combination of trametinib and dabrafenib, and (b) a secondary agent comprising L-buthionine-[S,R]-sulfoximine (BSO), or phenyl butyric acid (PBA) or a pharmaceutically acceptable salt thereof, and chloroquine or hydrochloroquine (HCQ), a container, and a package insert or label indicating the administration of the therapeutic combination for treating a hyperproliferative disorder.

In certain embodiments, the present invention provides a kit comprising a therapeutic combination consisting essentially of (a) an anti-cancer combination of vemurafenib and cobimetinib, or a combination of trametinib and dabrafenib, and (b) a secondary agent comprising L-buthionine-[S,R]-sulfoximine (B SO), or phenyl butyric acid (PBA) or a pharmaceutically acceptable salt thereof, and chloroquine or hydrochloroquine (HCQ), a container, and a package insert or label indicating the administration of the therapeutic combination for treating a hyperproliferative disorder.

In certain embodiments, the present invention provides a product comprising a therapeutic combination comprising (a) an anti-cancer combination of vemurafenib and cobimetinib, or a combination of trametinib and dabrafenib, and (b) a secondary agent comprising L-buthionine-[S,R]-sulfoximine (BSO), or phenyl butyric acid (PBA) or a pharmaceutically acceptable salt thereof, and chloroquine or hydrochloroquine (HCQ) as a combined preparation for separate, simultaneous or sequential use in the treatment of a hyperproliferative disorder.

In certain embodiments, the present invention provides a product comprising a therapeutic combination consisting essentially of (a) an anti-cancer combination of vemurafenib and cobimetinib, or a combination of trametinib and dabrafenib, and (b) a secondary agent comprising L-buthionine-[S,R]-sulfoximine (B SO), or phenyl butyric acid (PBA) or a pharmaceutically acceptable salt thereof, and chloroquine or hydrochloroquine (HCQ) as a combined preparation for separate, simultaneous or sequential use in the treatment of a hyperproliferative disorder.

In certain embodiments, the present invention provides a method for treating a hyperproliferative disorder in a mammal, comprising administering to the mammal a therapeutic combination comprising (a) an anti-cancer combination of vemurafenib and cobimetinib, or a combination of trametinib and dabrafenib, and (b) a secondary agent comprising L-buthionine-[S,R]-sulfoximine (BSO), or phenyl butyric acid (PBA) or a pharmaceutically acceptable salt thereof, and chloroquine or hydrochloroquine (HCQ) as a combined preparation for separate, simultaneous or sequential use in the treatment of a hyperproliferative disorder.

In certain embodiments, the present invention provides a method for treating a hyperproliferative disorder in a mammal, comprising administering to the mammal a therapeutic combination consisting essentially of (a) an anti-cancer combination of vemurafenib and cobimetinib, or a combination of trametinib and dabrafenib, and (b) a secondary agent comprising L-buthionine-[S,R]-sulfoximine (B SO), or phenyl butyric acid (PBA) or a pharmaceutically acceptable salt thereof, and chloroquine or hydrochloroquine (HCQ) as a combined preparation for separate, simultaneous or sequential use in the treatment of a hyperproliferative disorder.

In certain embodiments, the therapeutic combination is administered for more than a month.

In certain embodiments, the therapeutic combination is administered for more than a year.

In certain embodiments, the PBA or pharmaceutically acceptable salt thereof is administered at a dosage of at least 1500 mg/day.

In certain embodiments, the vemurafenib is administered at a recommended dosage of at least about 900 mg twice daily. In certain embodiments, the vemurafenib is administered at a dosage of at least about 900-2000 (e.g. 900, 950, 1000, 1050, 1100, 1150, 1200, 1250, 1300, 1350, 1400, 1450, 1500, 1550, 1600, 1650, 1700, 1750, 1800, 1850, 1900, 1950 or 2000) mg twice daily. In certain embodiments, the vemurafenib is administered at a recommended dosage of at least 960 mg twice daily.

In certain embodiments, the cobimetinib is administered at a recommended dosage of at least about 50 mg orally once daily for the first 21 days of each 28-day cycle until disease progression or unacceptable toxicity. In certain embodiments, the cobimetinib is administered at a recommended dosage of at least about 50-100 mg (e.g., 50, 55, 60, 65, 70, 75, 80, 85, 90, 95, or 100 mg) orally once daily for the first 21 days of each 28-day cycle until disease progression or unacceptable toxicity. In certain embodiments, the cobimetinib is administered at a recommended dosage of 60 mg orally once daily for the first 21 days of each 28-day cycle until disease progression or unacceptable toxicity.

In certain embodiments, the trametinib is administered at a recommended dosage of at least about 1 mg orally once daily as a single agent. In certain embodiments, the trametinib is administered at a recommended dosage of at least about 1-5 mg (e.g., 1.0, 1.5, 2.0, 2.5, 3.0, 3.5, 4.0, 4.5 or 5.0 mg) orally once daily as a single agent. In certain embodiments, the trametinib is administered at a recommended dosage of at least about 1-5 mg (e.g., 1.0, 1.5, 2.0, 2.5, 3.0, 3.5, 4.0, 4.5 or 5.0 mg) orally once daily in combination with dabrafenib at a dosage of at least about 50-200 (e.g., 50, 55, 60, 65, 70, 75, 80, 85, 90, 95, 100, 110, 120, 130, 140, 150, 160, 170, 180, 190, or 200) mg orally twice daily. In certain embodiments, the trametinib is administered at a recommended dosage of 2 mg orally once daily as a single agent or in combination with dabrafenib 150 mg orally twice daily.

In certain embodiments, the BSO is administered at a dosage of at least 1 g/m²/day and up to 20 g/m²/day (e.g., 1, 2, 3, 4, 5, 6, 7, 8, 9, 10, 11, 12, 13, 14, 15, 16, 17, 18, 19 or 20 g/m²/day).

In certain embodiments, the chloroquine or HCQ is administered at a dosage of at least 50 mg/day to a maximum of 600 mg/day (e.g., 50, 60, 70, 80, 90, 100, 150, 200, 250, 300, 350, 400, 450, 500, 550, or 600 mg/day)

In certain embodiments, the present invention provides a use of (a) an anti-cancer combination of vemurafenib and cobimetinib, or a combination of trametinib and dabrafenib, and (b) a secondary agent comprising L-buthionine-[S,R]-sulfoximine (BSO), or phenylbutyric acid (PBA) or a pharmaceutically acceptable salt thereof, and chloroquine or hydrochloroquine (HCQ) for the therapeutic treatment of a hyperproliferative disorder.

In certain embodiments, the hyperproliferative disorder is cancer.

In certain embodiments, the cancer is melanoma.

In certain embodiments, the anti-cancer combination is administered in combination with the secondary agent.

In certain embodiments, (a) the secondary agent is administered simultaneously with the anti-cancer combination; or (b) the secondary agent and the anti-cancer combination are administered sequentially; or (c) administration of the one or more anti-cancer combination begins about 1 to about 10 days before administration of the secondary agent; or (d) administration of secondary agent begins about 1 to about 10 days before administration of the anti-cancer combination; or (e) administration of the secondary agent and administration of the anti-cancer combination begins on the same day.

In certain embodiments, the BSO is administered in combination with vemurafenib and cobimetinib, and the cancer is melanoma.

In certain embodiments, the PBA and HCQ is administered in combination with vemurafenib and cobimetinib, and the cancer is melanoma.

In certain embodiments, the BSO is administered in combination with trametinib and dabrafenib, and the cancer is melanoma.

In certain embodiments, the PBA and HCQ is administered in combination with trametinib and dabrafenib, and the cancer is melanoma.

In certain embodiments, the present invention provides a method of depleting glutathione (GHS) in a cancer cell in a patient comprising administering to the patient buthionine sulfoximine (BSO), wherein the cancer cell is determined to be resistant to BRAF inhibitors (BRAFᵢ).

In certain embodiments, the cancer cell is also determined to be resistant to mitogen-activated protein kinase enzyme inhibitors (MEKᵢ).

In certain embodiments, the present invention provides a method of inhibiting endoplasmic reticulum (ER)-stress and autophagy in a cancer cell in a patient comprising administering to the patient buthionine sulfoximine (BSO), wherein the cancer cell is determined to be resistant to BRAF inhibitors (BRAFᵢ).

In certain embodiments, the cancer cell is also determined to be resistant to mitogen-activated protein kinase enzyme inhibitors (MEKᵢ).

In certain embodiments, the present invention provides a method of depleting glutathione (GHS) in a cancer cell in a patient comprising administering to the patient buthionine sulfoximine (BSO), wherein the cancer cell is determined to be resistant to mitogen-activated protein kinase enzyme inhibitors (MEKᵢ).

In certain embodiments, the present invention provides a method of inhibiting endoplasmic reticulum (ER)-stress and autophagy in a cancer cell in a patient comprising administering to the patient buthionine sulfoximine (BSO), wherein the cancer cell is determined to be resistant to mitogen-activated protein kinase enzyme inhibitors (MEKᵢ).

In certain embodiments of the methods described above, the composition does not significantly inhibit viability of comparable non-cancerous cells.

In certain embodiments of the methods described above, the tumor is reduced in volume by at least 10%. In certain embodiments, the tumor is reduced by any amount between 1-100%, (i.e., 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%).

Administration of a compound as a pharmaceutically acceptable acid or base salt may be appropriate. Examples of pharmaceutically acceptable salts are organic acid addition salts formed with acids which form a physiological acceptable anion, for example, tosylate, methanesulfonate, acetate, citrate, malonate, tartrate, succinate, benzoate, ascorbate, α-ketoglutarate, and α-glycerophosphate. Suitable inorganic salts may also be formed, including hydrochloride, sulfate, nitrate, bicarbonate, and carbonate salts.

Pharmaceutically acceptable salts may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

The anti-cancer agents and the secondary agents can be formulated as pharmaceutical compositions and administered to a mammalian host, such as a human patient in a variety of forms adapted to the chosen route of administration, i.e., orally or parenterally, by intravenous, intramuscular, topical or subcutaneous routes.

Thus, the present compounds may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of a given unit dosage form. The amount of active compound in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

The active compound may also be administered intravenously or intraperitoneally by infusion or injection. Solutions of the active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active compound in the required amount in the appropriate solvent with various other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, the present compounds may be applied in pure form, i.e., when they are liquids. However, it may be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Examples of useful dermatological compositions which can be used to deliver the compounds of formula Ito the skin are known to the art; for example, see Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508).

The dosage of the PBA or pharmaceutically acceptable salt thereof and the anti-cancer agent will vary depending on age, weight, and condition of the subject. Treatment may be initiated with small dosages containing less than optimal doses, and increased until a desired, or even an optimal effect under the circumstances, is reached. In general, the dosage is about 450-600 mg/kg/day in patients weighing less than 20 kg, or 9.9-13.0 g/m²/day in larger patients. Higher or lower doses, however, are also contemplated and are, therefore, within the confines of this invention. A medical practitioner may prescribe a small dose and observe the effect on the subject's symptoms. Thereafter, he/she may increase the dose if suitable. In general, the PBA or pharmaceutically acceptable salt thereof and the anti-cancer agent are administered at a concentration that will afford effective results without causing any unduly harmful or deleterious side effects, and may be administered either as a single unit dose, or if desired in convenient subunits administered at suitable times.

A pharmaceutical composition of the invention is formulated to be compatible with its intended route of administration. For example, the therapeutic agent may be introduced directly into the cancer of interest via direct injection. Additionally, examples of routes of administration include oral, parenteral, e.g., intravenous, slow infusion, intradermal, subcutaneous, oral (e.g., ingestion or inhalation), transdermal (topical), transmucosal, and rectal administration. Such compositions typically comprise the PBA or pharmaceutically acceptable salt thereof and the anti-cancer agent and a pharmaceutically acceptable carrier. As used herein, “pharmaceutically acceptable carrier” is intended to include any and all solvents, dispersion media, coatings, antibacterial and anti-fungal agents, isotonic and absorption delaying agents, and the like, compatible with pharmaceutical administration, and a dietary food-based form. The use of such media and agents for pharmaceutically active substances is well known in the art and food as a vehicle for administration is well known in the art.

Solutions or suspensions can include the following components: a sterile diluent such as water for injection, saline solution (e.g., phosphate buffered saline (PBS)), fixed oils, a polyol (for example, glycerol, propylene glycol, and liquid polyetheylene glycol, and the like), glycerine, or other synthetic solvents; antibacterial and antifungal agents such as parabens, chlorobutanol, phenol, ascorbic acid, thimerosal, and the like; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol or sorbitol, and sodium chloride in the composition. Prolonged administration of the injectable compositions can be brought about by including an agent that delays absorption. Such agents include, for example, aluminum monostearate and gelatin. The parenteral preparation can be enclosed in ampules, disposable syringes, or multiple dose vials made of glass or plastic.

It may be advantageous to formulate compositions in dosage unit form for ease of administration and uniformity of dosage. Dosage unit form as used herein refers to physically discrete units suited as unitary dosages for an individual to be treated; each unit containing a predetermined quantity of active compound calculated to produce the desired therapeutic effect in association with the required pharmaceutical carrier. The dosage unit forms of the invention are dependent upon the amount of a compound necessary to produce the desired effect(s). The amount of a compound necessary can be formulated in a single dose, or can be formulated in multiple dosage units. Treatment may require a one-time dose, or may require repeated doses.

“Systemic delivery,” as used herein, refers to delivery of an agent or composition that leads to a broad biodistribution of an active agent within an organism. Some techniques of administration can lead to the systemic delivery of certain agents, but not others. Systemic delivery means that a useful, preferably therapeutic, amount of an agent is exposed to most parts of the body. To obtain broad biodistribution generally requires a blood lifetime such that the agent is not rapidly degraded or cleared (such as by first pass organs (liver, lung, etc.) or by rapid, nonspecific cell binding) before reaching a disease site distal to the site of administration. Systemic delivery of lipid particles can be by any means known in the art including, for example, intravenous, subcutaneous, and intraperitoneal. In a preferred embodiment, systemic delivery of lipid particles is by intravenous delivery.

“Local delivery,” as used herein, refers to delivery of an active agent directly to a target site within an organism. For example, an agent can be locally delivered by direct injection into a disease site, other target site, or a target organ such as the liver, heart, pancreas, kidney, and the like.

The term “mammal” refers to any mammalian species such as a human, mouse, rat, dog, cat, hamster, guinea pig, rabbit, livestock, and the like.

The terms “treat” and “treatment” refer to both therapeutic treatment and prophylactic or preventative measures, wherein the object is to prevent or decrease an undesired physiological change or disorder, such as the development or spread of cancer. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

The invention will now be illustrated by the following non-limiting Example.

### EXAMPLE 1

### Simultaneous Targeting of Glutathione Dependent Activation of ER-Stress and Autophagy Inhibits Acquisition of Resistance to MAPKᵢₕ in Melanoma

Melanoma resistance to MAPK inhibitors (MAPKᵢₕ) has been attributed to drug induced activation of endoplasmic reticulum (ER)-stress responses that stimulate increased autophagic flux. In this example, it is shown that adaptation to MAPKᵢ is accompanied by 70% depletion of glutathione (GSH) and a 30-fold increase in oxidized glutathione (GSSG) indicating that MAPKᵢ are inducing profound thiol-mediated metabolic oxidative stress. It is further shown that pharmacologic depletion of glutathione (GSH) in melanoma cells, using GSH-synthesis inhibitor buthionine sulfoximine (BSO), completely inhibits acquisition of resistance to MAPKᵢ by preventing ER-stress responses and inhibiting autophagic flux. Finally, combining MAPKᵢ with simultaneous inhibition of autophagic flux and attenuation of ER-stress responses inhibited acquisition of MAPKᵢₕ resistance in melanoma tumors in mice and significantly enhanced overall survival (20% complete remissions). These results show that simultaneous targeting GSH metabolism, ER-stress, and autophagy provides a novel biochemical paradigm for abrogating the development of resistance to MAPKᵢₕ therapy in malignant melanoma.

Attempts to overcome melanoma resistance to MAPKᵢₕ by inhibiting autophagic flux or ER-stress pathways alone have been unsuccessful clinically. Here, GSH is shown to play a central role in activating these protective pathways, as well as providing resistance to BRAFi-induced oxidative stress. A GSH synthesis inhibitor (BSO) prevented acquisition of resistance to BRAF inhibitors (BRAFᵢₕ) in melanoma cells. Furthermore, MAPKᵢₕ combined with simultaneous inhibition of autophagic flux and attenuation of ER-stress can prevent the acquisition of resistance melanoma tumors in mice to MAPKᵢₕ. Thus, the results presented here provide new combination therapies for metastatic melanoma that can prevent the acquisition of resistance to MAPKᵢ and improve outcomes for metastatic melanoma patients.

Introduction

The introduction of drugs that inhibit the mitogen-activated protein kinase pathway (MAPKᵢₕ) represented a paradigm shift in treatment options for metastatic melanoma patients positive for BRAFV600x mutations (e.g., BRAFV600E). However, while initial response rates increased dramatically compared to previous chemotherapies, melanoma almost invariably (within months) developed resistance to BRAFᵢₕ and subsequently-introduced combination BRAFᵢₕ+MAPKᵢₕ therapies. Thus, reported 5-year progression-free survival rates of patients undergoing these treatments remain less than 25%, and no current clinical treatment option has been made available that overcomes the acquisition of drug resistance to MAPKᵢₕ in metastatic melanoma patients.

The precise mechanisms of melanoma adaptation to MAPKᵢₕ are not entirely understood. Plausible mechanisms include re-activation of the MAPK pathway via NRAS and MEK mutations; altered/alternative oncogenic pathways; copy number amplification of BRAFV600E; alternative splicing of BRAFV600E; the presence of subpopulations of resistant-melanoma stem-like cells; and metabolic reprogramming. Recently, emerging evidence has indicated a role for autophagy, and (in particular) autophagy initiated by activation of unfolded protein response (UPR) pathways in the adaptation of metastatic melanoma to MAPKᵢₕ. Furthermore, the induction of autophagy and the presence of ER-stress markers in BRAFV600E metastatic melanoma cells suggest that metabolic adaptations of these cells occur as compensatory survival mechanisms that involve mitochondrial metabolism, but the role of oxidative stress in this process is currently unknown. In this example, it is shown that adaptation to MAPKᵢₕ is accompanied by 68% depletion of glutathione (GSH) and a 30-fold increase in oxidized glutathione (GSSG) indicating that MAPK pathway inhibition (MAPKᵢ) is inducing profound thiol-mediated metabolic oxidative stress. It is further shown that depletion of glutathione (GSH) in melanoma cells, using GSH-synthesis inhibitor buthionine sulfoximine (BSO), completely inhibits acquisition of resistance to MAPKᵢₕ, ostensibly by preventing ER-stress responses and inhibiting autophagic flux. Finally, combining MAPKᵢₕ with a novel simultaneous inhibition of autophagic flux and attenuation of ER-stress responses inhibited acquisition of MAPKᵢₕ resistance in melanoma tumors in mice; significantly enhancing overall survival (20% complete remissions). These results support the simultaneous targeting of GSH metabolism, ER-stress, and autophagy to provide a novel biochemical paradigm for abrogating the development of resistance to MAPKᵢ therapy in malignant melanoma.

Results

Acquisition of Resistance to MAPKᵢₕ in Melanoma Cells is Mediated by Profound Thiol-Mediated Oxidative Stress and Can Be Prevented by Inhibiting Synthesis of Glutathione.

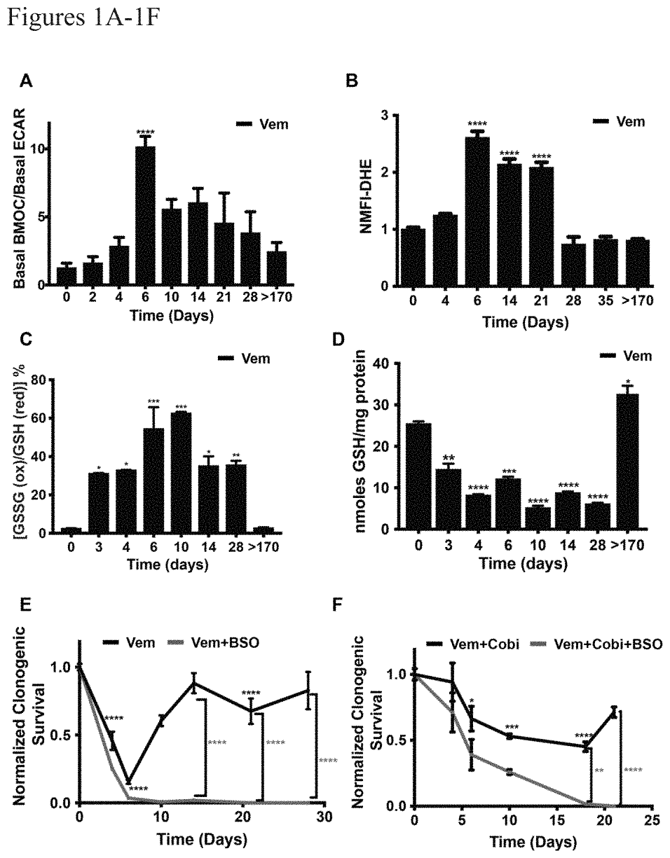

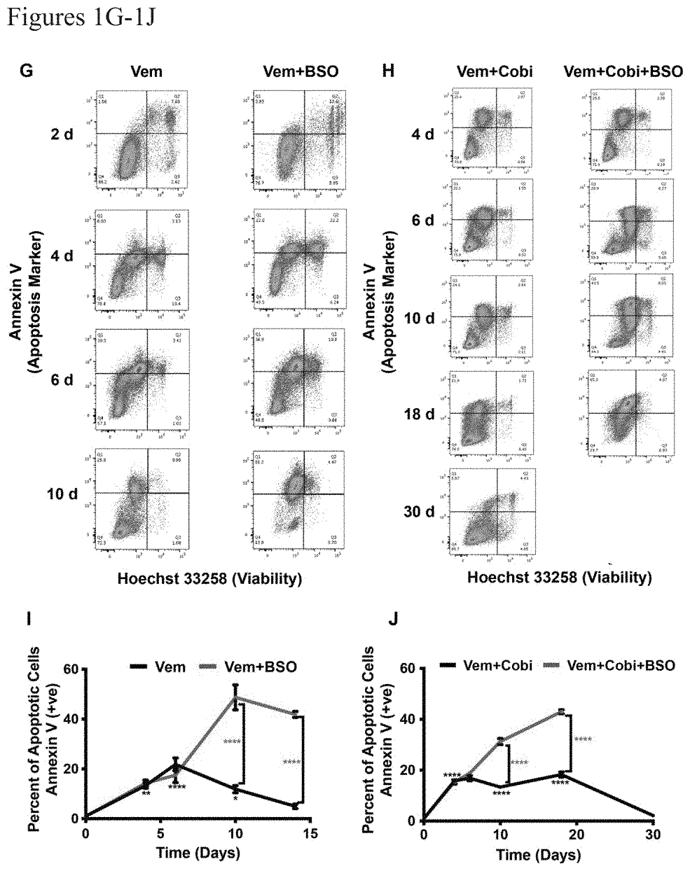

Initial response rates to MAPKᵢₕ (BRAFᵢₕ and BRAFᵢₕ+MEKᵢₕ) are often significant (and sometimes remarkable), but melanoma almost invariably (within months) develops resistance to these treatments. It has been further documented that continuous MAPKᵢ brings about changes in cellular metabolism accompanied with a shift in metabolism from glycolysis to oxidative phosphorylation in vitro. Changes were examined in metabolism and oxidative state of A375 BRAFV600E mutant melanoma cells from initiation of BRAFᵢₕ (Vem, 5 μM) through the acquisition of resistance (approximately 30 days). An initial increase in the ratio of oxygen consumption linked to basal mitochondrial respiration (OCR) and extracellular acidification rate (ECAR) was observed in A375 melanoma cells treated with BRAF inhibitor (BRAFᵢₕ) vemurafenib that plateaued within approximately 2 weeks of the initiation of treatment (FIG. 1A). The initial increase in OCR was followed by an eventual decrease in OCR to levels observed prior to the introduction of the BRAFᵢₕ. Changes in cellular metabolism coincided with a nearly identical pattern in the cellular redox state of the cell when quantified by the method of DHE oxidation by fluorescence intensity. Thus, an initial increase in the oxidation of DHE occurred in A375 cells in presence of Vem (5 μM) was observed to subside (with the acquisition of resistance; FIG. 1B). These findings suggest a connection between the cellular redox state of the cell and the acquisition of resistance (FIG. 1B). Considering the critical relationship between cellular responses to oxidative stress and the glutathione cellular redox buffer, the changes in glutathione (GSH) levels and the ratio of GSH to the oxidized form (GSH:GSSG) with prolonged MAPKᵢ in the A375 cells treated with Vem (5 μM) were evaluated over the same time period (FIG. 1D). These experiments revealed profound changes in these parameters with an observed 68% depletion of glutathione (GSH) and a 30-fold increase in oxidized glutathione (GSSG) (FIG. 1C). Furthermore, as the cells adapted to MAPKᵢ, the total GSH concentration re-equilibrated to levels comparable to untreated control cells (>170 d Vem treatment), ostensibly as adapted cells regained a homeostatic redox state.

To determine if the observed MAPKᵢ-induced GSH imbalance plays a causal role in development of resistance, clonogenic survival assays were conducted in which cells were treated with GSH-synthesis inhibitor buthionine sulfoximine (BSO) (0.5 mM) with and without Vem. BSO depletes cellular GSH by inhibiting GSH synthesis via competitive inhibition of gamma-glutamylcysteine synthetase. BSO alone did not affect the clonogenic survival of A375 melanoma cells. However, the combination of BSO with MAPKᵢₕ completely prevented the development of resistance of A375 cells to MAPKᵢ that was observed in A375 cells treated with Vem alone (FIG. 1E); and in combination with MEKᵢₕ Cobimetinib (Cobi, 0.1 μM) (FIG. 1F). These results were substantiated by analysis of Annexin V expression in A375 cells upon treatment with MAPKᵢₕ with and without the inclusion of BSO. These experiments revealed significant increases in the percent of apoptotic cells (Annexin V positive) observed when cells were treated with MAPKᵢₕ (Vem and Vem+Cobi) in combination with BSO (i.e., inhibition of GSH synthesis) (FIGS. 1G-J). These results suggest that MAPKᵢ-induces profound thiol-mediated oxidative stress that plays a key role in initiating acquisition of resistance to MAPKᵢₕ.

Prolonged MAPKᵢ Induces Protective ER-Stress Responses in Melanoma Cells that Can Be Attenuated by Inhibiting Synthesis of Glutathione.

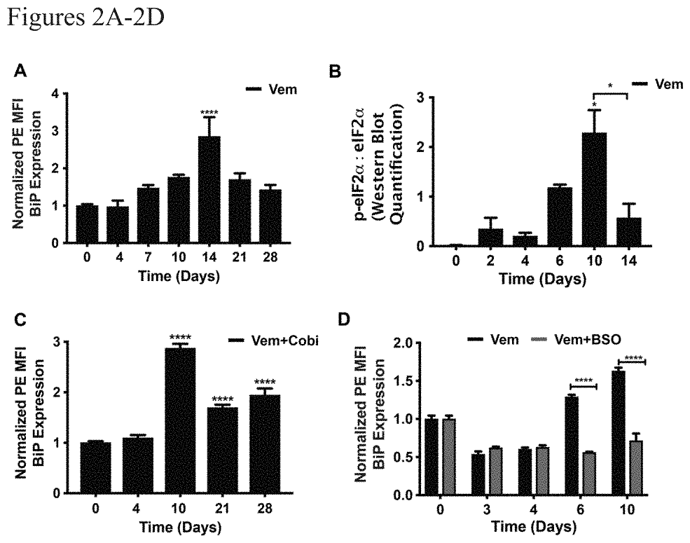

The GSH/GSSG ratio is known to be a fundamental buffering parameter of the ER, and ER-stress responses have been implicated in the initiation of autophagy and acquisition of resistance to MAPKᵢₕ. Thus, the impact of MAPKᵢ-induced GSH imbalance on the expression of ER-stress response proteins was assessed. Changes in UPR markers GRP78 (BiP) and eI2Fa (phospho-eI2Fa to inactivated eI2Fa) were determined by western blot in A375 cells treated with Vem alone and in combination with Cobi (FIGS. 2A-C). It was observed that continuous MAPKᵢ results in a gradual increase in the expression of ER-stress markers, however (in a nearly identical pattern observed with changes in metabolic parameters) the initial increase was followed by a decrease to pre-treatment homeostatic levels as the cells adapted to MAPKᵢ. To explore the role of MAPKᵢ-induced GSH imbalance on activation of UPR, similar time-course experiments were performed to monitor the expression of UPR markers with continuous MAPKᵢ with and without GSH depletion (using BSO). Depletion of the GSH pool using BSO in this way rendered the cells incapable of initiating the ER-stress response and no increase in the expression of GRP78 was seen when A375 cells were treated continuously with Vemurafenib in the presence of BSO (FIG. 2D). These results support the hypothesis that GSH plays a key role in enabling the adaptation of melanoma cells to MAPKᵢ via mediating activation of UPR.

Prolonged MAPKᵢ Induces Increases in Autophagic Flux in Melanoma Cells that Can Be Diminished by Inhibiting Synthesis of Glutathione.

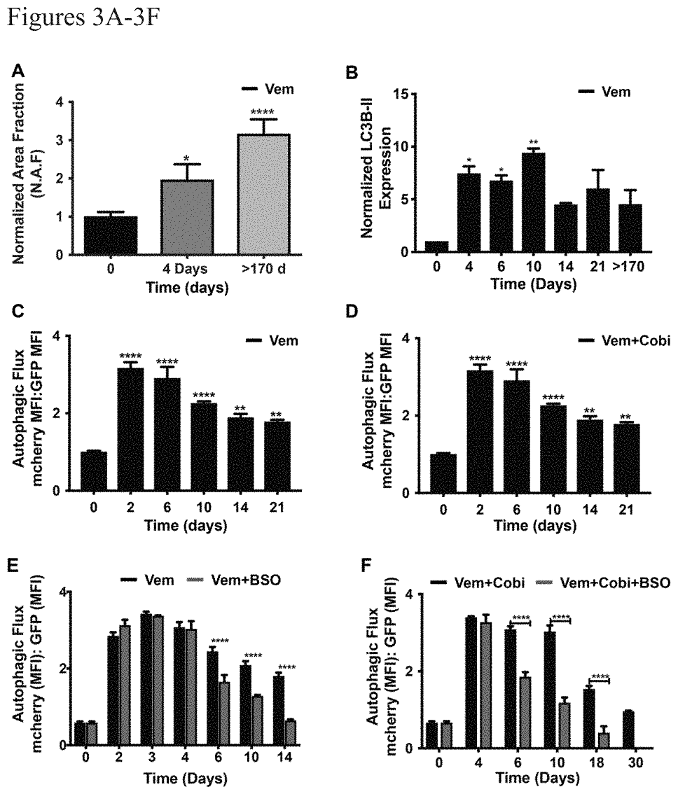

UPR responses signal the activation of cytoprotective pathways such as autophagy. The role of GSH in mediating the acquisition of resistance was further explored by examining autophagy and autophagic flux responses to MAPKᵢ in A375 metastatic melanoma cells. Initial observations revealed a significant increase in the area fraction of cells covered by autophagosomes (obtained by transmission electron microscopy (TEM)) increased following treatment with MAPKᵢₕ (Vem; 5 μM) (FIG. 3A). In parallel experiments, significant increases in the expression of autophagic flux marker LC3B-II were observed with continuous MAPKᵢ by Vem in A375 cells (FIG. 3B). Because autophagy is a dynamic process, autophagic flux was measured using A375-CG-LC3B cells (a cell line that was constructed by stably transfecting A375 with LC3B that possesses a tandem labeling of EGFP and mcherry fluorescent tags.

Briefly, A375-CG-LC3B cells were obtained using a retroviral vector containing a coding sequence for a mCherry-EGFP-LC3B tandem protein (pBABE-puro mCherry-EGFP-LC3B, #22418, Addgene). The retrovirus was packaged in GP2-293 cells by co-transfection of 1 μg of viral DNA constructs and 1 μg of envelope plasmid (pVSV-G) in 20 μL PolyFect transfection reagent (Qiagen) for 48 hours at 37° C., 5% CO₂. Virus-rich media was collected, aliquoted, and stored at −80° C. Cells were infected using the virus-rich media in DMEM (high glucose) for 12 h followed by incubation in complete media (high-glucose DMEM with 10% FBS) for 24 h. Infected cells were selected using 2 μg mL⁻¹ puromycin and transfection was confirmed using microscopy and flow cytometry. A375-CG-LC3B cells were maintained in high glucose DMEM (Gibco) supplemented with 10% FBS (Gibco) and 1% Penstrep and 1 μg mL⁻¹ puromycin. All the cell lines were maintained in 5% CO₂ at 37° C. Autophagy was quantified as autophagic flux (A_{f}) using A375-CG-LC3B cells.

A_{f}=F_{mcherry}/F_{GFP}

Where F_{mcherry} is mcherry fluorescence measured using the 532 nm laser and F_{GFP} is GFP fluorescence measured using the 488 nm laser on LSR II. The cells were plated in six-well plates (80,000 cells well⁻¹), that were treated in triplicate when cell confluency reached 60-80%. At specified time points, the total cell pool was collected, centrifuged (1200 rpm, 5 min), and washed with cold PBS twice. The cell pellets were stained with apoptotic marker Annexin V and Hoechst 33258 as described above. Samples were analyzed on an LSR II flow cytometer (BD). A_{f} was calculated for live, non-apoptotic (Hoechst negative, Annexin V negative) population using the derived parameter function in Flow Jo software (V10.4).

Briefly, fusion of the lysosome to the autophagosome results in an observable decrease in pH of the autolysosomal compartment that is intended to promote hydrolysis of encapsulated cellular debris. While the fluorescence of mCherry is not affected by the decrease in pH that occurs when lysosomes fuse with autophagosomes, EGFP fluorescence is degraded. Thus, cells with higher autophagic flux are less green due to fusion of autophagosomes with lysosomes, which increases the mCherry:EGFP ratio in the cell. Under conditions of continuous MAPKᵢ with Vem alone (FIG. 3C) and in combination with Cobimetinib (Cobi, 0.1 μM) (FIG. 3D) increased autophagic flux (which remained elevated even when the cells became resistant to MAPKᵢ) was observed. To assess the role of MAPKᵢ-induced GSH imbalance in the initiation of increased autophagy, autophagic flux was thus monitored with continuous MAPKᵢ with Vem alone and Vem+Cobi, with and without BSO (FIGS. 3E-F). It was observed that depletion of total GSH using BSO significantly decreases autophagic flux induced by MAPKᵢ. These results further show that GSH imbalance plays a key role in the development of resistance to MAPKᵢₕ via activation of ER-stress responses that mediate protective pathways including autophagy.

Simultaneous Attenuation of BRAFᵢ-Induced ER-Stress Responses and Autophagic Flux in Melanoma Cells Abrogates Acquisition of Resistance to BRAFᵢ in Melanoma Cells.

ER-stress mediated autophagy has been implicated in the acquisition of resistance to MAPKᵢₕ in metastatic melanoma. However, clinical trials exploring the potential of autophagic flux inhibitors alone as a means to overcome resistance have proven largely unsuccessful. Results presented herein suggest that drug-induced cytoprotective ER-stress responses and increases in autophagic flux may act independently to establish the resistant phenotype through a common thiol dependent pathway. To explore the role ER-Stress and autophagy play in the development of resistance to MAPKᵢₕ in melanoma cells A375 melanoma cells were treated simultaneously with 4-phenylbutyrate (PBA; a known ER-stress attenuator) and Hydroxychloroquine (HCQ; a known inhibitor of autophagy) in combination with MAPKᵢₕ.

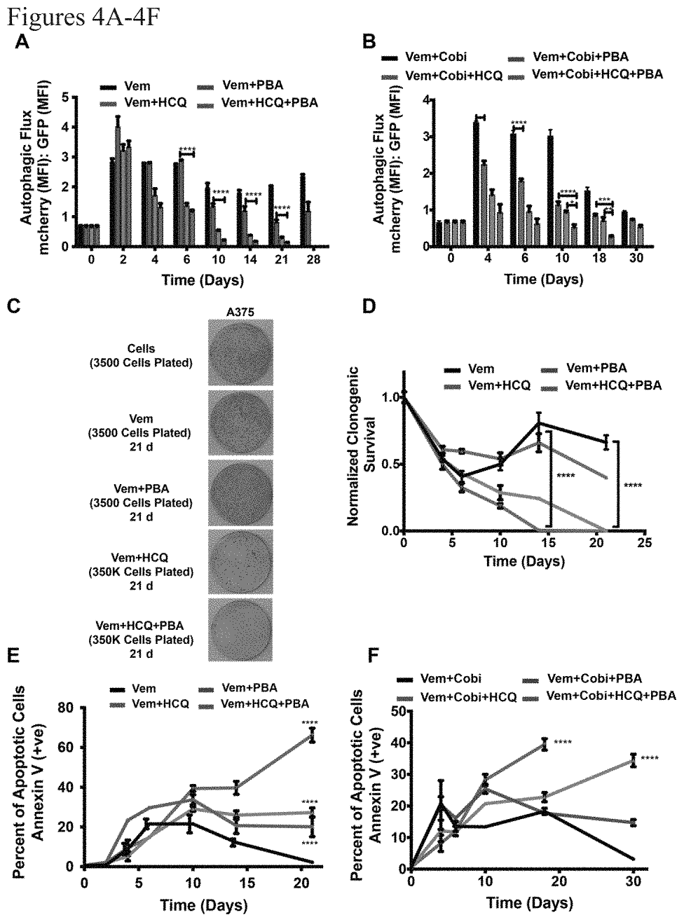

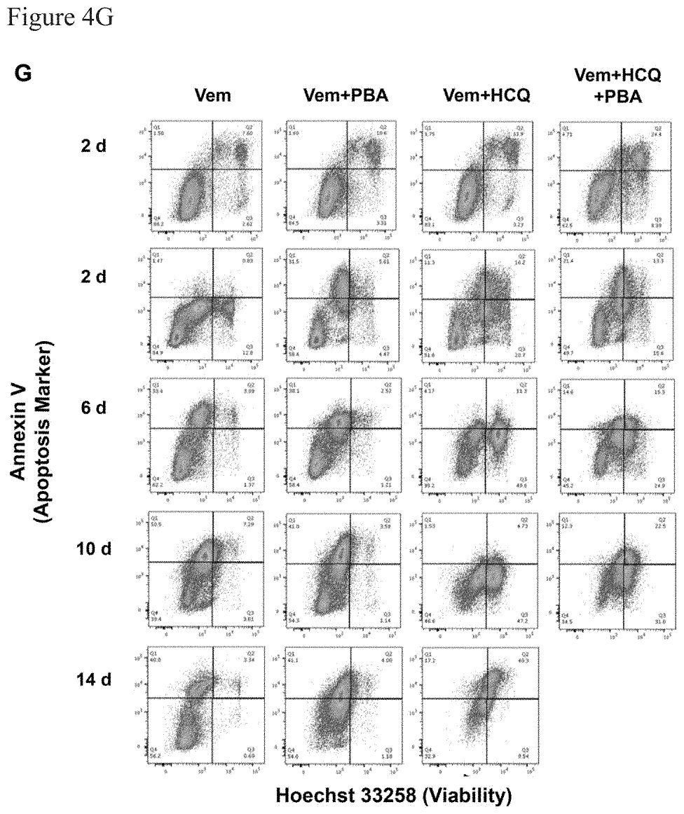

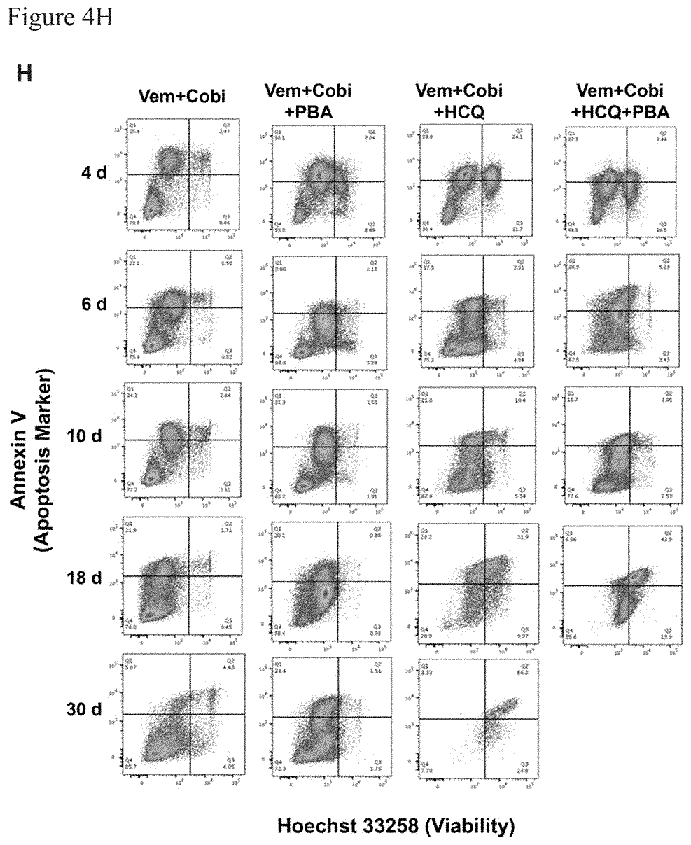

Changes in autophagic flux were quantified with the development of drug resistance in A375-CG-LC3B cells at pre-determined time points within the timeframe of observed acquisition of resistance to MAPKᵢ. In these experiments, A375 cells were treated with MAPKᵢₕ alone (black bar); in combination with only PBA (green bar); only HCQ (brown bar); and both PBA and HCQ (simultaneous inhibition of autophagic flux and attenuation of ER-stress) (red bar). Predictably, it was observed that MAPKᵢ with Vem alone (FIG. 4A) and in combination with Cobimetinib (Cobi, 0.1 μM) (FIG. 4B) increased the autophagic flux, which remained higher than untreated naive control cells even when the cells became resistant to MAPKᵢₕ. Further, a decrease in autophagic flux was observed when cells were treated with a combination of MAPKᵢₕ and PBA suggesting inhibition of UPR-mediated autophagy. However, the presence of a significant autophagic flux in the presence of PBA alone indicated a role of other signaling pathways in the activation of autophagy. Treatment of cells with HCQ decreased autophagic flux, but the combination of PBA and HCQ with MAPKᵢₕ completely diminished autophagic flux. Furthermore, the decrease in the autophagic flux observed with simultaneous treatment of A375 cells with the MAPKᵢₕ+PBA and HCQ combination completely prevented acquisition of drug resistance as quantified by clonogenic survival of cells (FIGS. 4C-D). Furthermore, the percent of apoptotic population was significantly higher when cells were treated with the combination of MAPKᵢₕ (Vem and Vem+Cobi), PBA and HCQ (FIGS. 4E-F). Additionally, it was observed that the simultaneous inhibition of autophagic flux and ER-stress (PBA and HCQ) in the presence of MAPKᵢₕ shifted the adapting cell population towards cell death and apoptosis more significantly than individual inhibition with Vem alone (FIG. 4G) and in combination with Cobimetinib (Cobi, 0.1 μM) (FIG. 4H). These results suggest that simultaneous inhibition of MAPKᵢ-induced ER-stress and autophagy has the potential to prevent resistance to MAPKᵢₕ in A375 melanoma cells.

Pharmacologically Inhibiting Autophagy and ER-Stress Simultaneously in Combination with MAPKᵢₕ Improves Overall Survival in Melanoma Tumor-Bearing Mice.

To further explore the potential of simultaneous pharmacological inhibition of ER-stress and autophagy in preventing resistance to MAPKᵢₕ, A375 xenograft bearing mice (FIGS. 5A-D) were treated with MAPKᵢₕ or MAPKᵢₕ in combination with PBA (250 mg kg-1 per dose, twice a day, i.p.) and HCQ (10 mg kg-1 per dose, twice per day, oral gavage). Clinical trials have established the superiority of BRAFᵢₕ (Vem) and MAPKᵢₕ (cobi) combination over administration of BRAFᵢₕ alone. The experimental groups included MAPKᵢ by Vem (BRAFᵢₕ) (16 mg kg-1 per dose, twice a day, oral gavage) alone and in combination with Cobi (MAPKᵢₕ) (1 mg kg-1 per dose, once a day, oral gavage). In these experiments, the observed average rate of tumor growth in untreated control mice is similar to mice treated with Vem alone (FIG. 5A), indicating resistance to BRAFᵢₕ. Predictably, the rate of tumor growth is slower in mice treated with the combination of Vem+Cobi (FIG. 5B), compared to Vem alone. Nonetheless, tumors continued to grow. Simultaneous inhibition of ER-stress and autophagy by treatment with the combination of PBA and HCQ had no effect of tumor growth (FIG. 5A), although a slight increase in survival was observed in combination with Vem (FIG. 5C). On the other hand, the combination of PBA and HCQ with Vem+Cobi significantly decreased rate of tumor growth (FIG. 5B) and significantly increased survival (FIG. 5D).

The potential for simultaneous inhibition of ER-stress and autophagy in overcoming resistance to MAPKᵢₕ was further explored using a 451LuBR (BRAFᵢ resistant melanoma) cell-line xenograft mouse model treated in the same way. Predictably, administering Vem alone had no effect of tumor growth and overall survival, establishing that these tumors are resistant to BRAFᵢ. Somewhat surprisingly, administration of Vem+PBA and Vem+HCQ resulted in increases in tumor growth rate (FIG. 5E) and a decrease in median survival (compared to control) (FIG. 5F), indicating that individual inhibition of autophagy and attenuation of ER-stress potentially enhance tumor growth. However, simultaneous inhibition of ER-stress and autophagy with PBA and HCQ in the presence of Vem resulted in reduced rate of tumor growth (FIG. 5E, brown dotted line) and a significantly higher median survival (FIG. 5F, brown dotted line) (20% complete response, 20% partial response).

Discussion

Melanoma is one of the fastest growing cancer incidences in the United States, and is the third most common type of skin cancer with over 90,000 diagnoses expected in 2018. Surgery combined with radiation can be curative at early stages, but metastatic melanoma is considered a lethal disease. The introduction of drugs that inhibit the mitogen-activated protein kinase pathway (MAPKᵢ) represented a paradigm shift in treatment options for metastatic melanoma patients positive for BRAFV600x mutations (e.g., BRAFV600E). However, while initial response rates increased dramatically compared to previous chemotherapies, melanoma almost invariably (within months) developed resistance to BRAFᵢₕ alone, as well as to combination BRAFᵢₕ+MEKᵢₕ combination therapies introduced more recently. While several plausible explanations for the acquisition of resistance to MAPKᵢ have been suggested, emerging evidence has implicated ER-stress mediated autophagy as a mechanism of resistance that can be targeted for drug therapy. However, the underlying mechanisms that initiate these pathways are not known.

The MAPK pathway is known to regulate cellular and mitochondrial metabolism by regulating expression of glycolysis-related genes and mitochondrial biogenesis/function regulating factors (MITF and PGC1a). Furthermore, studies have shown that BRAFᵢ induces an increase in oxidative phosphorylation (OXPHOS) by mediating upregulation of MITF and PGC1a and reducing the conversion of glucose to lactate (aerobic glycolysis), thereby re-routing glucose metabolism from glycolysis to OXPHOS. Because mitochondria are the primary source of ROS in cells, changes in mitochondrial function are likely to alter the levels of cellular and mitochondrial ROS. Increased cellular ROS in melanoma cells has been implicated in disease progression.

A recent study showed an increase in production of cellular and mitochondrial ROS within hours of the initiation of MAPKᵢ in BRAFV600E melanoma cells. In this Example, it is shown that a steep increase in oxygen consumption rate (and Mitosox oxidation) occurs in cells treated continuously with MAPKᵢ that peaks (at approximately 10 days) and decreases to pretreatment homeostatic levels as the cells become resistant to MAPKᵢ (FIGS. 1A, 1B). The crosstalk between cellular redox state and antioxidants is well established and changes in the cellular redox state affect the cellular antioxidant machinery. Of the constituents of the antioxidant repertoire of the cell, glutathione (GSH) is an essential protective cellular antioxidant.

In addition to maintaining cellular redox balance, GSH also plays a pivotal role in cellular signaling and protein folding in the ER. Significantly, the present data show that MAPKᵢ induces a 68% decrease in total GSH and a 30-fold increase in the ratio GSSG/GSH (FIG. 1C, 1D). Similar to the trends observed in OCR and oxidative state of the cells, GSH and GSSG:GSH peak within the same time period (6-10 days) and return to levels observed in untreated cells as melanoma cells become resistant to MAPKᵢ. These data indicate that the induced GSSG:GSH imbalance facilitates the acquisition of resistance in melanoma cells to MAPKᵢₕ via activation of cytoprotective pathways (UPR and autophagy; FIGS. 2, 3).

To test the hypothesis that GSH plays a key role in activation of cyrpoprotective pathways in response to MAPKᵢ, treatments were combined with GSH synthesis inhibitor buthionine sulfoximine to pharmacologically deplete total GSH levels in cells undergoing MAPKᵢ treatment. The results demonstrate that combining MAPKᵢₕ with an inhibitor of GSH synthesis (i.e., depletion of GSH levels using BSO) prevents acquisition of resistance to MAPKᵢ in melanoma cells and supports the involvement of thiol metabolism in the acquisition of resistance (FIG. 1E,J). BSO has been used in Phase 1 clinical trials in combination with chemotherapeutics such as melphalan in children with recurrent neuroblastoma. Additionally, a pilot single agent study has been completed in patients with myeloblastic leukemia. A current phase 1 clinical trial is examining the potential of BSO in combination with melphalan in patients with locally advanced malignant melanoma (NCT00661336). These phase 1 clinical trials are demonstrating that BSO can be well-tolerated patients.

ER-stress responses have been implicated in the acquisition of resistance to MAPKᵢ. Data presented herein substantiate that MAPKᵢ induces ER-stress responses that are implicated in the adaptive responses of melanoma cells toward the resistant phenotype and that these responses can be diminished by depleting cells of GSH using GSH synthesis inhibitor BSO (FIG. 2D). The ER plays a vital role in protein-folding in eukaryotic cells and thiols (e.g., GSH) play a key role in the protein folding machinery. An experiment to elucidate the central role of GSH was conducted using N-acetylecysteine as a source of thiols. In the ER, nascent proteins rely on the highly-regulated formation of disulfide bonds to form stable and functional proteins. GSH and GSSG act as a primary buffer that maintains the redox balance of ER lumen. An appropriate oxidative state of the ER lumen is essential for efficient formation of disulfide bonds and protein folding. A normal functioning ER lumen is significantly more oxidizing (GSH/GSSG range −1:1 to 3:1) than the overall cell (GSH/GSSG range—30:1 to 100:1) and is highly sensitive to changes in oxidative state of the cell. As a result, protein folding efficiency can be significantly disrupted by a small cellular glutathione imbalance.

When increased production of ROS exceeds the buffering capacity of the ER lumen, the protein folding machinery is disrupted, leading to a series of downstream ER-stress responses referred to collectively as the unfolded protein response (UPR). The UPR invokes several downstream signaling pathways via ER-resident ROS sensors (PERK, IRE1a, and ATF6) that guide the cell towards restoration of homeostasis or (potentially) apoptosis. Here it was shown that continuous MAPKᵢ induces ER-stress (UPR) responses that peak coincident with changes in OCR and DHE oxidation (FIGS. 1A, B), as well as profound decreases in GSH and elevation of the GSSG:GSH ratio (FIG. 1C, D). Taken together, these data demonstrate a pivotal role for GSH in the acquisition of resistance to MAPKᵢ in metastatic melanoma.

Autophagy is known to be an integral part of normal cellular function, but can also play a dual role—at once acting as a tumor suppressor in benign and early-stage tumors including melanoma, while facilitating tumor growth and resistance in advanced cancers. Chloroquine and HCQ are FDA-approved antimalarial drugs, which are known to inhibit autophagy by preventing lysosomal fusion to autophagosomes. While HCQ is reported to be well tolerated, inhibition of autophagy in combination with mTOR inhibitors and alkylating chemotherapies have produced mixed results. For example, while partial response and stable disease were observed in only 14% and 27% of metastatic melanoma treated with the combination of HCQ and temozolomide (TMZ, an alkylating agent), 2 of 6 patients with refractory BRAF wildtype melanoma had near complete response, and prolonged stable disease respectively. On the other hand, the median progression-free survival in 13 melanoma patients treated with HCQ (1200 mg d⁻¹) in combination with temsirolimus (an mTOR inhibitor) was 3.5 months in a Phase 2 trial (N=13) with clinical evidence of autophagic flux inhibition in tumor biopsies.60 Pathology analysis of tumor samples collected in mouse studies presented herein showed lower levels of autophagy markers in mouse tumors that responded to MAPKᵢ/HCQ combination treatments. These findings and observations suggest that alternative pathways actively circumvent the cytotoxic effects of autophagy inhibition. To date, no clinical trial has evaluated the response of melanoma subjects treated with the combination of MAPKᵢₕ and inhibitors of autophagy. Data presented herein suggest that MAPKᵢ-induced ER-stress responses play a role in the resistant phenotype.

In the in vivo setting, data presented herein further established a role of ER-stress responses to induce increases in autophagic flux in the acquisition of resistance by attenuating ER-stress responses using 4-phelybutyric acid (PBA). PBA is a multifunctional aromatic fatty acid that has been shown to attenuate ER-stress by acting as a non-specific chaperone of misfolded proteins, as well as possessing histone deacetylase inhibitor (HDACi) activity by virtue of its physical structure (carboxy terminus connected via an alkane linker to a lipophilic opposing moiety). Phase 1 and Phase 2 clinical trials focused on the use of PBA as a HDACi have shown that PBA can be safely administered to human patients. While no complete response or partial remission was seen in these clinical trials, stable disease has been observed and PBA is known to be tolerated at very high doses (up to 27 g per day) in patients suffering from urea cycle disorders.61-63 To date, no clinical study has evaluated the role of PBA in combination with an autophagy inhibitor to overcome drug resistance in melanoma. Data presented herein suggest that combining MAPKᵢ with PBA and HCQ to simultaneously attenuate ER-stress responses and inhibit autophagic flux (FIG. 4A, 4B) has the potential to be a clinically relevant approach to improving outcomes for metastatic melanoma patients.

### SUMMARY AND CONCLUSIONS

In summary, the findings and observations herein led to the idea that MAPKᵢ induces metabolic oxidative stress that is plays a role initiating ER-stress responses that further lead to increases in autophagic flux (and subsequent acquisition of resistance to MAPKᵢₕ in metastatic melanoma). Initially, the investigation focused on changes in oxidative metabolism (OCR) and the oxidative state of melanoma cells (via DHE oxidation assay) with prolonged MAPKᵢ. It was observed that (initially) MAPKᵢ induced a significant increase in OCR over approximately 10 days of continuous MAPKᵢ (FIG. 1A). However, as melanoma cells adapted to MAPKᵢ, OCR peaked and inflected toward decreased levels that were similar to levels observed in untreated melanoma cells. These changes in OCR were accompanied with an identical pattern in oxidative state (by DHE oxidation; FIG. 1B) of the melanoma cells when treated with MAPKᵢₕ. Considering the key role that GSH plays in maintaining a healthy redox environment in the cell (and ER) the potential role that GSH and the GSH:GSSG ratio play in initiating ER-stress responses (FIG. 2.2) that lead to autophagic flux increases (FIG. 2.3) that convey resistance was examined. These investigations showed that adaptation of melanoma cells to MAPKᵢ is accompanied by a 68% depletion of glutathione (GSH) and a 30-fold increase in oxidized glutathione (GSSG), indicating that MAPKᵢₕ are inducing profound thiol-mediated metabolic oxidative stress. It was further shown that pharmacologic depletion of glutathione (GSH) in melanoma cells, using GSH-synthesis inhibitor buthionine sulfoximine (BSO), completely inhibits acquisition of resistance to MAPKᵢ by preventing ER-stress responses (FIG. 2D) and inhibiting autophagic flux (FIG. 3E, 3F). Finally, combining MAPKᵢₕ with simultaneous inhibition of autophagic flux and attenuation of ER-stress responses inhibited acquisition of MAPKᵢₕ resistance in melanoma tumors in mice and significantly enhanced overall survival (20% complete remissions) (FIG. 5F). These results show that simultaneous targeting GSH metabolism, ER-stress, and autophagy provides a novel biochemical paradigm for abrogating the development of resistance to MAPKᵢ therapy in malignant melanoma.

### EXAMPLE 2

### Thiol-Redox Imbalance Mediates Development of MAPKih-Resistance via Autophagy in Metastatic Melanoma

BRAF regulates cellular oxidative metabolism in melanoma cells. However, the role of MAPKi-mediated changes in oxidative metabolism in development of resistance is not known.

The clinical impact of MAPK inhibitors (MAPKi) on patient survival has been limited by the acquisition of resistance to these drugs. Increased autophagic flux has been implicated in resistance, but the underlying mechanism has yet to be identified. It is shown that resistance to MAPKi is accompanied by profound alterations in thiol-redox state and oxidative metabolism in cells that lead to increased autophagic flux (and resistance). It is further shown that inhibition of glutathione (GSH) synthesis using GSH synthase inhibitor buthionine sulfoximine (B SO) in combination with MAPKi inhibited development of resistance by preventing increased autophagic flux. This effect was reversed by increasing reduced thiols using β-mercaptoethanol (ME). Pharmacological inhibition of autophagic flux directly (using hydroxychloroquine) prevented resistance in vitro and significantly enhanced overall survival in vivo (20% complete remissions in mice). Furthermore, inhibiting GSH synthesis (using BSO) in combination with MAPKi led to 75% complete remissions in mice and significantly improved overall survival.

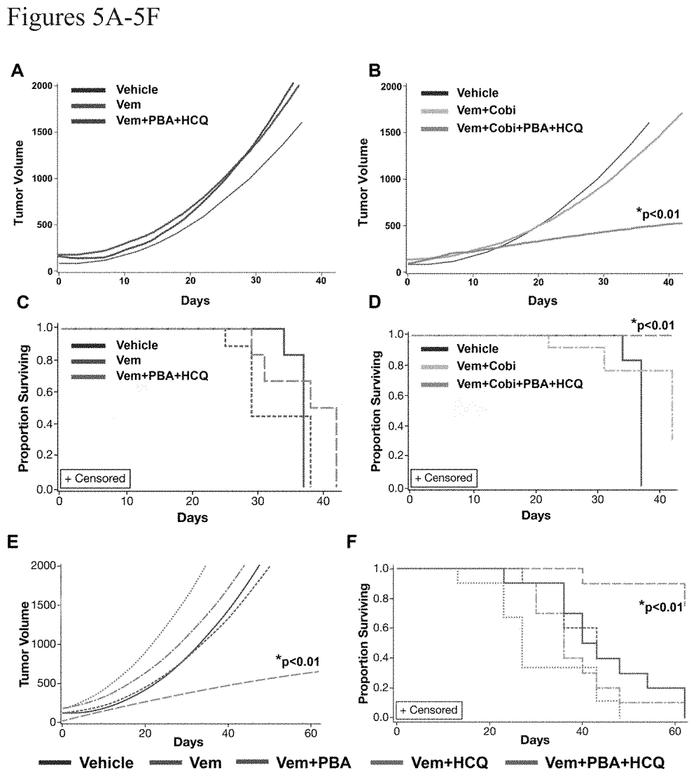

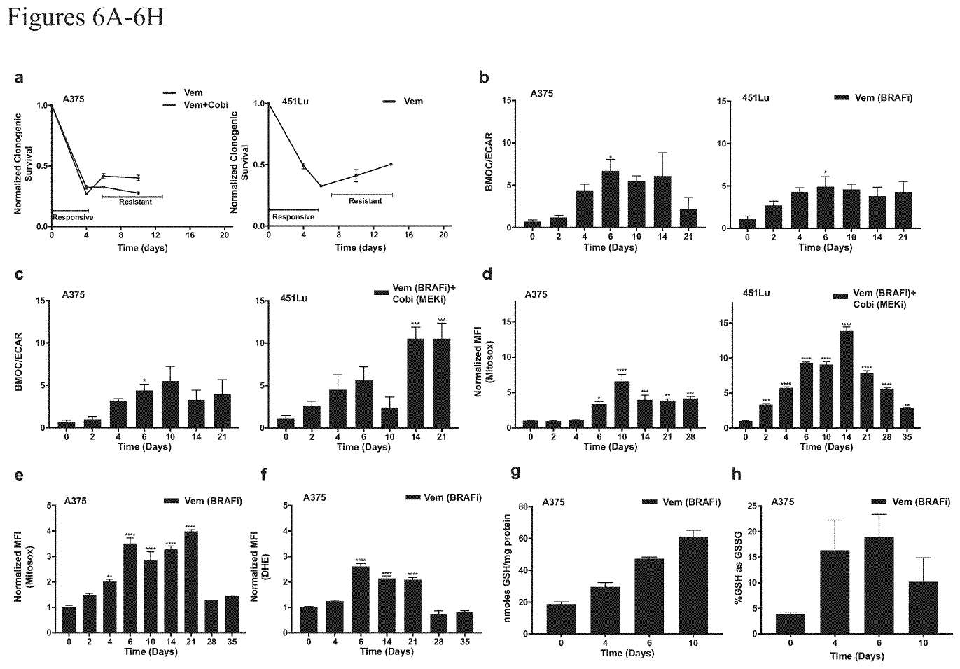

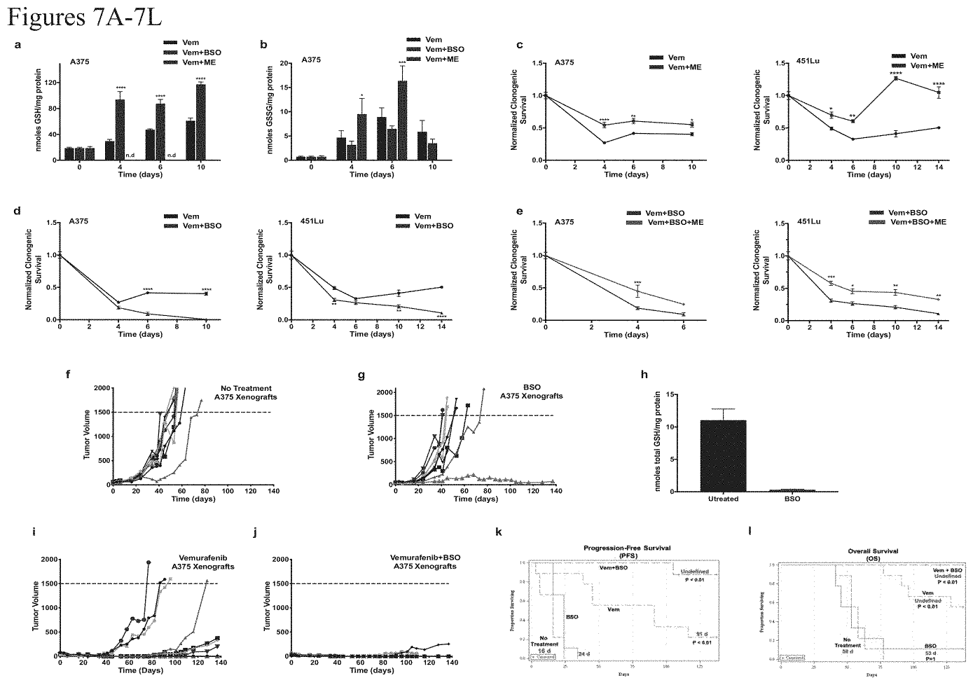

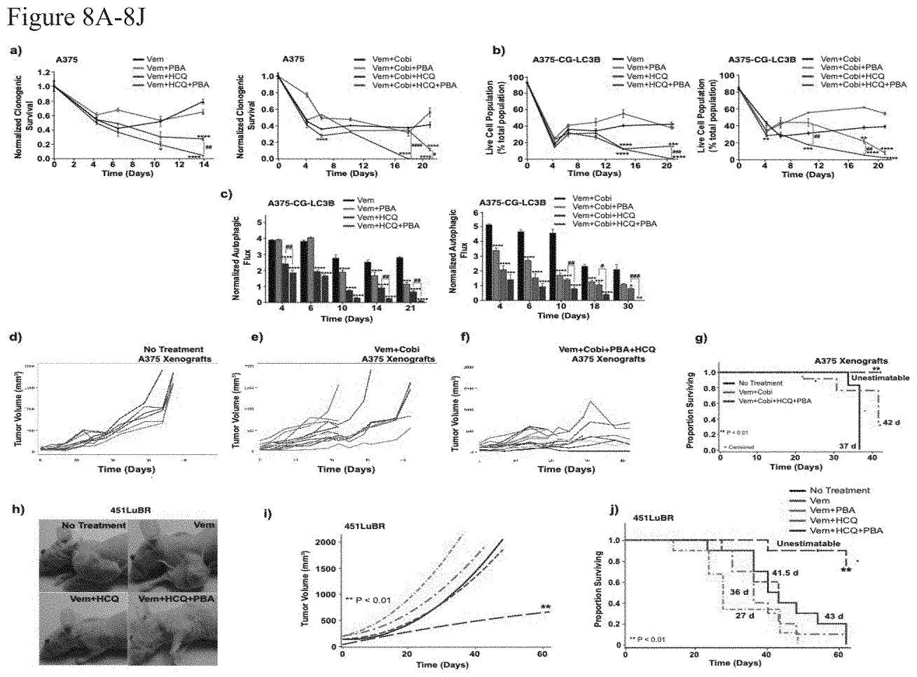

Acquisition of resistance to MAPKi was accompanied by changes in oxidative metabolism of melanoma cells. FIGS. 6A-6H. Reduced thiols played a vital role in development of resistance to MAPKi. FIGS. 7A-7L. Simultaneous inhibition of autophagic flux and attenuation of UPR prevented development of resistance to MAPKi in vivo and in vitro. FIGS. 8A-8J. Thus, a link between MAPKi mediated thiol-redox imbalance and resistance has been identified.

Although the foregoing specification and example fully disclose and enable certain embodiments, they are not intended to limit the scope, which is defined by the claims appended hereto.

All publications, patents and patent applications are incorporated herein by reference. While in the foregoing specification certain embodiments have been described, and many details have been set forth for purposes of illustration, it will be apparent to those skilled in the art that additional embodiments and certain details described herein may be varied considerably without departing from basic principles.

The use of the terms “a” and “an” and “the” and similar referents are to be construed to cover both the singular and the plural, unless otherwise indicated herein or clearly contradicted by context.

The terms “comprising,” “having,” “including,” and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the technology and does not pose a limitation on the scope of the technology unless otherwise claimed. No language in the specification should be construed as indicating any non-claimed element as essential to the practice of the technology.

Throughout this specification, unless the context requires otherwise, the word “comprise” or variations such as “comprises” or “comprising”, will be understood to imply the inclusion of a stated integer or group of integers but not the exclusion of any other integer or group of integers. It is also noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the embodiment.

Embodiments are described herein, including the best mode known to the inventors. Variations of those embodiments may become apparent to those of ordinary skill in the art upon reading the foregoing description. The inventors expect skilled artisans to employ such variations as appropriate, and the inventors intend for the embodiments to be practiced otherwise than as specifically described herein. Accordingly, this technology includes all modifications and equivalents of the subject matter recited in the claims appended hereto as permitted by applicable law. Moreover, any combination of the above-described elements in all possible variations thereof is encompassed by embodiments unless otherwise indicated herein or otherwise clearly contradicted by context.

1. A therapeutic regimen comprising (a) an anti-cancer BRAF inhibitor agent or combination of BRAF inhibitor and MEK inhibitor, and (b) a secondary agent comprising L-buthionine-[S,R]-sulfoximine (BSO), or combination of phenylbutyric acid (PBA) or a pharmaceutically acceptable salt thereof, and chloroquine or hydrochloroquine (HCQ) for the therapeutic treatment of a hyperproliferative disorder.

2. The therapeutic regimen of claim 1, wherein the anti-cancer agent consists essentially of a BRAF inhibitor agent.

3. The therapeutic regimen of claim 1, wherein the anti-cancer agent consists essentially of a combination of a BRAF inhibitor agent and a MEK inhibitor.

4. The therapeutic regimen of claim 1, wherein the BRAF inhibitor agent is vemurafenib or dabrafenib.

5. The therapeutic regimen of claim 1, wherein the MEK inhibitor is cobimetinib or trametinib.

6. The therapeutic regimen of claim 1, consisting essentially of (a) an anti-cancer combination of vemurafenib and cobimetinib, or a combination of trametinib and dabrafenib, and (b) a secondary agent comprising L-buthionine-[S,R]-sulfoximine (BSO), or combination of phenylbutyric acid (PBA) or a pharmaceutically acceptable salt thereof, and chloroquine or hydrochloroquine (HCQ) for the therapeutic treatment of a hyperproliferative disorder.

7. The therapeutic regimen of claim 1, wherein the hyperproliferative disorder is cancer.

8. The therapeutic regimen of claim 7, wherein the cancer is melanoma.

9-17. (canceled)

18. The therapeutic regimen of claim 1, wherein the anti-cancer combination comprises vemurafenib and cobimetinib.

19. The therapeutic regimen of claim 1, wherein the anti-cancer combination comprises trametinib and dabrafenib.

20. The therapeutic regimen of claim 1, wherein the secondary agent comprises BSO.

21. The therapeutic regimen of claim 1, wherein the secondary agent comprises PBA or a pharmaceutically acceptable salt thereof and chloroquine or HCQ.

22. (canceled)

23. The therapeutic regimen of claim 1, wherein vemurafenib, cobimetinib, PBA and HCQ are administered in combination, and the cancer is melanoma.

24. The therapeutic regimen of claim 1, wherein vemurafenib, cobimetinib, and BSO are administered in combination, and the cancer is melanoma.

25. The therapeutic regimen of claim 1, wherein trametinib, dabrafenib, PBA and HCQ are administered in combination, and the cancer is melanoma.

26. The therapeutic regimen of claim 1, wherein trametinib, dabrafenib, and BSO are administered in combination, and the cancer is melanoma.

27-36. (canceled)

37. A method for treating a hyperproliferative disorder in a mammal, comprising administering to the mammal a therapeutic combination comprising (a) an anti-cancer combination of vemurafenib and cobimetinib, or a combination of trametinib and dabrafenib, and (b) a secondary agent comprising L-buthionine-[S,R]-sulfoximine (BSO), or phenyl butyric acid (PBA) or a pharmaceutically acceptable salt thereof, and chloroquine or hydrochloroquine (HCQ) as a combined preparation for separate, simultaneous or sequential use in the treatment of a hyperproliferative disorder.

38-62. (canceled)

63. A method of depleting glutathione (GHS) or inhibiting endoplasmic reticulum (ER)-stress and autophagy in a cancer cell in a patient comprising administering to the patient buthionine sulfoximine (BSO), wherein the cancer cell is determined to be resistant to BRAF inhibitors (BRAFᵢ) or is determined to be resistant to mitogen-activated protein kinase enzyme inhibitors (MEKi).

64. The method of claim 63, wherein the cancer cell is determined to be resistant to BRAF inhibitors (BRAFᵢ) and is determined to be resistant to mitogen-activated protein kinase enzyme inhibitors (MEKᵢ).

65-66. (canceled)

67. The method claim 63, wherein the cancer cell is determined to be resistant to mitogen-activated protein kinase enzyme inhibitors (MEKᵢ).

68. (canceled)
